SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, Homo sapiens [sequence omitted]

SEQ ID NO: 5 - protein, 107 aa, Homo sapiens [sequence omitted]

SEQ ID NO: 6 - protein, 5 aa, Homo sapiens
SFAMS

SEQ ID NO: 7 - protein, 17 aa, Homo sapiens
AISGSGGGTYYADSVKG

SEQ ID NO: 8 - protein, 13 aa, Homo sapiens
DKILWFGEPVFDY

SEQ ID NO: 9 - protein, 11 aa, Homo sapiens
RASQSVSSYLA

SEQ ID NO: 10 - protein, 7 aa, Homo sapiens
DASNRAT

SEQ ID NO: 11 - protein, 10 aa, Homo sapiens
QQRSNWPPTF

SEQ ID NO: 12 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Homo sapiens
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, Homo sapiens
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, Homo sapiens
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, Homo sapiens
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, Homo sapiens
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, Homo sapiens [sequence omitted]

SEQ ID NO: 21 - protein, 107 aa, Homo sapiens [sequence omitted]

SEQ ID NO: 22 - protein, 447 aa, Homo sapiens [sequence omitted]

(19) United States
(12) Patent Application Publication
(10) Pub. No.: US 2023/0233676 A1
(43) Pub. Date: Jul. 27, 2023

(54) Use of Approved Anti-CD38 Antibody Drug Product in Combination with Pomalidomide and Dexamethasone to Treat Multiple Myeloma

(71) Applicant: Janssen Biotech, Inc., Horsham, PA (US)

(72) Inventor: Ming Qi, Phoenixville, PA (US)

(21) Appl. No.: 18/100,056

(22) Filed: Jan. 23, 2023

Related U.S. Application Data
(60) Provisional application No. 63/302,303, filed on Jan. 24, 2022.

(51) Int. Cl. A61K 39/395; A61P 35/00; A61K 38/47; A61K 31/454; A61K 31/573; G06Q 30/06

(52) U.S. Cl. CPC A61K 39/39558; A61P 35/00; A61K 38/47; C12Y 302/01035; A61K 31/454; A61K 31/573; G06Q 30/06; A61K 2039/545

(57) ABSTRACT

The present invention relates to methods of treating multiple myeloma using an approved drug product comprising daratumumab and hyaluronidase in combination with pomalidomide and dexamethasone. Also described are methods for improving progression-free survival in multiple myeloma patients and methods of selling or offering for sale a drug product comprising daratumumab and hyaluronidase.

### CROSS REFERENCE TO RELATED APPLICATIONS

This application claims the benefit of U.S. Provisional Application Ser. No. 63/302,303, filed 24 Jan. 2022 and U.S. Provisional Application Ser. No. 63/302,746, filed 25 Jan. 2022. The entire contents of each of the aforementioned applications is incorporated herein by reference in their entirety.

### REFERENCE TO SEQUENCE LISTING SUBMITTED ELECTRONICALLY

This application contains a sequence listing, which is submitted electronically via The United States Patent and Trademark Center Patent Center as an XML formatted sequence listing with a file name “JBI6698USNP1 Sequence Listing.xml” and a creation date of 6 Jan. 2023, and having a size of 23 Kb. The sequence listing submitted via Patent Center is part of the specification and is herein incorporated by reference in its entirety.

### FIELD OF THE INVENTION

The present invention relates to methods of treating multiple myeloma using an approved drug product comprising daratumumab and hyaluronidase in combination with pomalidomide and dexamethasone. Also described are methods of selling or offering for sale an approved drug product comprising daratumumab and hyaluronidase for the treatment of multiple myeloma.

### BACKGROUND OF THE INVENTION

B-cell malignancies include B-cell chronic lymphocytic leukemia, mantle cell lymphoma, Burkitt lymphoma, follicular lymphoma, diffuse large B-cell lymphoma, multiple myeloma, Hodgkin's lymphoma, hairy cell leukemia, primary effusion lymphoma and AIDS-related Non-Hodgkin's Lymphoma. B-cell malignancies comprise more than 85% of diagnosed lymphomas.

Multiple myeloma (MM) is characterized by the latent accumulation of secretory plasma cells in bone marrow with a low proliferative index and an extended life span. The disease ultimately attacks bones and bone marrow, resulting in multiple tumors and lesions throughout the skeletal system. Approximately 1% of all cancers and slightly more than 10% of all hematologic malignancies can be attributed to multiple myeloma. Incidence of multiple myeloma increases in the aging population, with the median age at time of diagnosis being about 61 years.

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., 14:1284-92, 2012; Chiarugi et al., Nature Reviews 12:741-52, 2012). In addition to signaling via Ca²⁺, CD38 signaling occurs via crosstalk with antigen-receptor complexes on T- and B-cells or other types of receptor complexes, e.g., MHC molecules, involving CD38 in several cellular responses, but also in switching and secretion of IgG1. CD38 is expressed on various malignant cells.

Currently recognized treatments for MM include various chemotherapeutic agents with or without autologous stem cell transplantation. The chemotherapeutic agents include combination regimens of glutamic acid derivatives, proteasome inhibitors and steroids including bortezomib, lenalidomide, thalidomide and dexamethasone as well as melphalan with or without daratumumab. However, the disease remains incurable. Thus, there is a need for additional therapeutics for MM.

### SUMMARY OF THE INVENTION

The invention provides a method of treating MM comprising administering an approved drug product containing daratumumab and hyaluronidase to an adult patient with MM in an amount that is described in a drug product label for the drug product.

The invention also provides a method of selling an approved drug product comprising daratumumab and hyaluronidase, the method comprising selling such drug product, wherein a drug product label for a reference listed drug for such drug product includes instructions for treating MM.

The invention also provides a method of offering for sale an approved drug product comprising daratumumab and hyaluronidase, the method comprising offering for sale such drug product, wherein a drug product label for a reference listed drug for such drug product includes instructions for treating MM.

The invention also provides a method of selling an approved drug product comprising daratumumab and hyaluronidase, the method comprising selling such drug product, wherein the drug product label for a reference listed drug for such drug product comprises PFS data.

The invention also provides a method of offering for sale an approved drug product comprising daratumumab and hyaluronidase, said method comprising offering for sale such drug product, wherein the drug product label for a reference listed drug for such drug product comprises PFS data.

The invention also provides a method of improving PFS in an adult patient with MM, the method comprising administering to the adult patient an approved drug product comprising daratumumab and hyaluronidase.

### BRIEF DESCRIPTION OF THE DRAWINGS

FIG. 1 shows the Kaplan-Meier curve for progression-free survival (PFS) for DARZALEX FASPRO with pomalidomide and dexamethasone (DARZALEX FASPRO-Pd) versus pomalidomide and dexamethasone (Pd) alone.

### DETAILED DESCRIPTION OF THE INVENTION

All publications, including but not limited to patents and patent applications, cited in this specification are herein incorporated by reference as though fully set forth.

“DARZALEX FASPRO” is a sterile, preservative-free, colorless to yellow, and clear to opalescent solution for subcutaneous injection use supplied as individually packaged single-dose vials providing 1,800 mg of daratumumab and 30,000 units of hyaluronidase per 15 mL.

“CD38” refers to the human CD38 protein (synonyms: ADP-ribosyl cyclase 1, cADPr hydrolase 1, cyclic ADP-ribose hydrolase 1). Human CD38 has an amino acid sequence shown in GenBank accession number NP_001766 and in SEQ ID NO: 1. It is well known that CD38 is a single pass type II membrane protein with amino acid residues 1-21 representing the cytosolic domain, amino acid residues 22-42 representing the transmembrane domain, and residues 43-300 representing the extracellular domain of CD38.

SEQ ID NO: 1
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J Exp Med 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol Biol 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

“Drug product” or “approved drug product” is product that contains an active pharmaceutical ingredient that has been approved for marketing for at least one indication by a governmental authority, e.g., the Food and Drug Administration or the similar authority in other countries. In the present invention, DARZALEX FASPRO is an approved drug product. The approved drug product could also be a biosimilar drug product, a Biologic License Application drug product or a supplemental Biologic License Application drug product

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A multispecific antibody, such as a bispecific antibody or a trispecific antibody is included in the term monoclonal antibody.

“Recombinant” includes antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“In combination with” means that two or more therapeutics are administered to a subject together in a mixture, concurrently as single agents or sequentially as single agents in any order.

“Pharmaceutical composition” refers to a product that results from combining an anti-CD38 antibody and a hyaluronidase and includes both fixed and non-fixed combinations. Pharmaceutical composition typically includes a pharmaceutically acceptable carrier. “Fixed combinations” refers to a single pharmaceutical composition comprising the anti-CD38 antibody and the hyaluronidase administered simultaneously in the form of a single entity or dosage.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

“Progression-free survival” or “PFS” is the length of time during and after treatment of a disease, such as multiple myeloma, that a patient lives with the disease, but the disease does not progress or get worse. In the case of multiple myeloma, disease progression is defined according to the International Myeloma Working Group diagnostic criteria.

“Treat” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or disease well as those subjects prone to have the physiological change or disease.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

### Pharmaceutical Compositions

The invention provides a pharmaceutical composition comprising an anti-CD38 antibody and a hyaluronidase.

The pharmaceutical composition is useful for subcutaneous administration of the anti-CD38 antibody to a subject in need of anti-CD38 antibody therapy, such as a subject having a cancer, for example a CD38-positive hematological malignancy. Without wishing to be bound by any particular theory, subcutaneous administration of the anti-CD38 antibody may have reduced infusion related reactions when compared to the intravenous administration of the anti-CD38 antibody.

In some embodiments, the pharmaceutical composition is a fixed combination.

In some embodiments, the pharmaceutical composition is a non-fixed combination.

In some embodiments, the hyaluronidase is rHuPH20 having the amino acid sequence of SEQ ID NO: 22, namely residues 36-482 of wild type human hyaluronidase.

rHuPH20 is a recombinant hyaluronidase (HYLENEX® recombinant) and is described in Int. Pat. Publ. No. WO2004/078140.

Hyaluronidase is an enzyme that degrades hyaluronic acid (EC 3.2.1.35) and lowers the viscosity of hyaluronan in the extracellular matrix, thereby increasing tissue permeability.

Enzymatic activity of hyaluronidase, including rHuPH20 can be defined by units per mL (U/mL) or by total enzyme activity in a particular formulation (U).

The standard definition for one unit (U) of enzyme activity is the amount of enzyme that catalyzes the reaction of 1 nmol of substrate per minute.

In some embodiments, the anti-CD38 antibody in the pharmaceutical composition competes for binding to CD38 with an antibody comprising a heavy chain variable region (VH) of SEQ ID NO: 4 and a light chain variable region (VL) of SEQ ID NO: 5.

In some embodiments, the anti-CD38 antibody in the pharmaceutical composition binds at least to the region SKRNIQFSCKNIYR (SEQ ID NO: 2) and the region EKVQTLEAWVIHGG (SEQ ID NO: 3) of human CD38 (SEQ ID NO: 1).

In some embodiments, the anti-CD38 antibody in the pharmaceutical composition comprises a heavy chain complementarity determining region 1 (HCDR1), a HCDR2, a HCDR3, a light chain complementarity determining region 1 (LCDR1), a LCDR2 and a LCDR3 of SEQ ID NOs: 6, 7 and 8, 9, 10 and 11, respectively.

In some embodiments, the anti-CD38 antibody in the pharmaceutical composition comprises the VH of SEQ ID NO: 4 and the VL of SEQ ID NO: 5.

In some embodiments, the anti-CD38 antibody in the pharmaceutical composition comprises a heavy chain of SEQ ID NO: 12 and a light chain of SEQ ID NO: 13.

SEQ ID NO: 2
SKRNIQFSCKNIYR
SEQ ID NO: 3
EKVQTLEAWVIHGG
SEQ ID NO: 4
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSS
SEQ ID NO: 5
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK
SEQ ID NO: 6
SFAMS
SEQ ID NO: 7
AISGSGGGTYYADSVKG
SEQ ID NO: 8
DKILWFGEPVFDY
SEQ ID NO: 9
RASQSVSSYLA
SEQ ID NO: 10
DASNRAT
SEQ ID NO: 11
QQRSNWPPTF
SEQ ID NO: 12
EVQLLESGGGLVQPGGSLRLSCAVSGFTFNSFAMSWVRQAPGKGLEWVSAISGSGGGT
YYADSVKGRFTISRDNSKNTLYLQMNSLRAEDTAVYFCAKDKILWFGEPVFDYWGQGT
LVTVSSASTKGPSVFPLAPSSKSTSGGTAALGCLVKDYFPEPVTVSWNSGALTSGVHTFP
AVLQSSGLYSLSSWTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 13
EIVLTQSPATLSLSPGERATLSCRASQSVSSYLAWYQQKPGQAPRLLIYDASNRATGIPA
RFSGSGSGTDFTLTISSLEPEDFAVYYCQQRSNWPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 14
QVQLVQSGAEVKKPGSSVKVSCKASGGTFSSYAFSWVRQAPGQGLEWMGRVIPFLGIA
NSAQKFQGRVTITADKSTSTAYMDLSSLRSEDTAVYYCARDDIAALGPFDYWGQGTLV
TVSSAS
SEQ ID NO: 15
DIQMTQSPSSLSASVGDRVTITCRASQGISSWLAWYQQKPEKAPKSLIYAASSLQSGVPS
RFSGSGSGTDFTLTISSLQPEDFATYYCQQYNSYPRTFGQGTKVEIK
SEQ ID NO: 16
EVQLVQSGAEVKKPGESLKISCKGSGYSFSNYWIGWVRQMPGKGLEWMGIIYPHDSDA
RYSPSFQGQVTFSADKSISTAYLQWSSLKASDTAMYYCARHVGWGSRYWYFDLWGRG
TLVTVSS
SEQ ID NO: 17
EIVLTQSPATLSLSPGERATLSCRASQSVSSYLAWYQQKPGQAPGLLIYDASNRASGIPA
RFSGSGSGTDFTLTISSLEPEDFAVYYCQQRSNWPLTFGGGTKVEIK
SEQ ID NO: 18
QVQLVESGGGLVQPGGSLRLSCAASGFTFSSYYMNWVRQAPGKGLEWVSGISGDPSNT
YYADSVKGRFTISRDNSKNTLYLQMNSLRAEDTAVYYCARDLPLVYTGFAYWGQGTL
VTVSS
SEQ ID NO: 19
DIELTQPPSVSVAPGQTARISCSGDNLRHYYVYWYQQKPGQAPVLVIYGDSKRPSGIPER
FSGSNSGNTATLTISGTQAEDEADYYCQTYTGGASLVFGGGTKLTVLGQ
SEQ ID NO 20:
QVQLVQSGAEVAKPGTSVKLSCKASGYTFTDYWMQWVKQRPGQGLEWIGT
IYPGDGDTGYAQKFQGKATLTADKSSKTVYMHLSSLASEDSAVYYCARGD
YYGSNSLDYWGQGTSVTVSS
SEQ ID NO: 21:
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGG
GTKLEIK

The invention also provides a pharmaceutical composition comprising an anti-CD38 antibody comprising the VH of SEQ ID NO: 4 and the VL of SEQ ID NO: 5 and a hyaluronidase rHuPH20 of SEQ ID NO: 22, wherein the anti-CD38 antibody concentration in the pharmaceutical composition is about 120 mg/mL.

The invention also provides a pharmaceutical composition comprising an anti-CD38 antibody comprising the HCDR1, the HCDR2 and the HCDR3 of SEQ ID NOs: 6, 7 and 8, respectively, and the LCDR1, the LCDR2 and the LCDR3 of SEQ ID NOs: 9, 10 and 11, respectively and the hyaluronidase rHuPH20 of SEQ ID NO: 22, wherein the anti-CD38 antibody concentration in the pharmaceutical composition is about 120 mg/mL.

The invention also provides a pharmaceutical composition comprising about 1,800 mg of the anti-CD38 antibody comprising the VH of SEQ ID NO: 4 and the VL of SEQ ID NO: 5, and about 30,000 U of the hyaluronidase rHuPH20 of SEQ ID NO: 22, wherein the anti-CD38 antibody concentration in the pharmaceutical composition is about 120 mg/mL.

SEQ ID NO: 22
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQIFY

The pharmaceutical compositions of the invention further comprise a pharmaceutically acceptable carrier. Exemplary pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

The invention also provides a pharmaceutical composition comprising about 120 mg/mL of the anti-CD38 antibody comprising the VH of SEQ ID NO: 4 and the VL of SEQ ID NO: 5 and 2000 U/mL rHuPH20 (SEQ ID NO: 22) in about 10 mM L-histidine, 300 mM sorbitol, 0.04% w/v polysorbate-20 (PS-20), and about 1 mg/mL L-Methionine, pH 5.6.

The formulations to be used for in vivo administration are generally sterile. Sterility may be readily accomplished, e.g., by filtration through sterile filtration membranes.

### Methods of Treatment

The invention provides methods of treating multiple myeloma (MM) with an approved drug product.

The anti-CD38 antibody used in the methods of treatment of the invention is daratumumab. Daratumumab comprises the heavy chain variable region (VH) and the light chain variable region (VL) amino acid sequences shown in SEQ ID NOs: 4 and 5, respectively, the HCDR1, the HCDR2 and the HCDR3 of SEQ ID NOs: 6, 7 and 8, respectively, the LCDR1, the LCDR2 and the LCDR3 of SEQ ID NOs: 9, 10 and 11, respectively, and is of IgG1/x subtype and described in U.S. Pat. No. 7,829,693. Daratumumab heavy chain amino acid sequence is shown in SEQ ID NO: 12 and light chain amino acid sequence shown in SEQ ID NO: 13.

The hyaluronidase used in the methods of the invention is rHuPH20 having the amino acid sequence of SEQ ID NO: 22, namely residues 36-482 of wild type human hyaluronidase. rHuPH20 is a recombinant hyaluronidase (HYLENEX® recombinant) and is described in Int. Pat. Publ. No. WO2004/078140.

Hyaluronidase is an enzyme that degrades hyaluronic acid (EC 3.2.1.35) and lowers the viscosity of hyaluronan in the extracellular matrix, thereby increasing tissue permeability.

Enzymatic activity of hyaluronidase, including rHuPH20 can be defined by units per mL (U/mL) or by total enzyme activity in a particular formulation (U).

The standard definition for one unit (U) of enzyme activity is the amount of enzyme that catalyzes the reaction of 1 nmol of substrate per minute.

The invention also provides a method of treating MM, comprising administering to a subject in need thereof an approved drug product comprising the anti-CD38 antibody comprising the VH of SEQ ID NO: 4 and the VL of SEQ ID NO: 5 and the hyaluronidase rHuPH20 (SEQ ID NO: 22) subcutaneously for a time sufficient to treat the subject, wherein the anti-CD38 antibody concentration in the approved drug product is about 120 mg/mL.

The invention also provides a method of treating MM, comprising administering to a subject in need thereof an approved drug product comprising about 1,800 mg of the anti-CD38 antibody comprising the VH of SEQ ID NO: 4 and the VL of SEQ ID NO: 5, and about 30,000 U of the hyaluronidase rHuPH20 (SEQ ID NO: 22) for a time sufficient to treat the subject, wherein the anti-CD38 antibody concentration in the pharmaceutical composition is about 120 mg/mL.

The invention also provides a method of treating MM, comprising administering to a subject in need thereof an approved drug product comprising about 120 mg/mL of the anti-CD38 antibody comprising the VH of SEQ ID NO: 4 and the VL of SEQ ID NO: 5, and 2000 U/mL rHuPH20 (SEQ ID NO: 22) in about 10 mM L-histidine, about 300 mM sorbitol, about 0.04% w/v polysorbate-20 (PS-20), and about 1 mg/mL L-Methionine, about pH 5.6 for a time sufficient to treat the subject.

The anti-CD38 antibodies in the pharmaceutical compositions of the invention may induce killing of CD38-expressing tumor cells by antibody-dependent cell-mediated cytotoxicity (ADCC), antibody-dependent cellular phagocytosis (ADCP), complement dependent cytotoxicity (CDC), apoptosis, or modulation of CD38 enzymatic activity. The anti-CD38 antibodies in the pharmaceutical compositions of the invention may also mediate anti-tumor efficacy by their immunomodulatory effects by inducing CD4⁺ and CD8⁺ T cell proliferation, and/or by relieving inhibition of inflammatory responses mediated by myeloid-derived suppressor cells (MDSCs) and regulatory T cells (Tregs).

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells. For example, NK cells express FcγRIIIa, whereas monocytes express FcγRI, FcγRII and FcvRIIIa. Death of the antibody-coated target cell, such as CD38-expressing cells, occurs as a result of effector cell activity through the secretion of membrane pore-forming proteins and proteases. To assess ADCC activity of an antibody that specifically binds CD38, the antibody may be added to CD38-expressing cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include Tregs or MDSCs expressing CD38. In an exemplary assay, target cells are labeled with 20 μCi of ⁵¹Cr for 2 hours and washed extensively. Cell concentration of the target cells may be adjusted to 1×10⁶ cells/ml, and anti-CD38 antibodies at various concentrations are added. Assays are started by adding target cells at an effector:target cell ratio of 40:1. After incubation for 3 hr at 37° C. assays are stopped by centrifugation, and ⁵¹Cr release from lysed cells are measured in a scintillation counter. Percentage of cellular cytotoxicity may be calculated as % maximal lysis which may be induced by adding 3% perchloric acid to target cells.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using Tregs or MDSCs expressing CD38 as target cells engineered to express GFP or other labeled molecule. Effctor:target cell ratio may be for example 4:1. Effector cells may be incubated with target cells for 4 hours with or without anti-CD38 antibody. After incubation, cells may be detached using accutase. Macrophages may be identified with anti-CD11b and anti-CD14 antibodies coupled to a fluorescent label, and percent phagocytosis may be determined based on % GFP fluorescent in the CD11⁺CD14⁺ macrophages using standard methods.

“Complement-dependent cytotoxicity”, or “CDC”, refers to a mechanism for inducing cell death in which an Fc effector domain of a target-bound antibody binds and activates complement component C1q which in turn activates the complement cascade leading to target cell death. Activation of complement may also result in deposition of complement components on the target cell surface that facilitate ADCC by binding complement receptors (e.g., CR3) on leukocytes.

### Combination Therapies

The pharmaceutical composition of the methods of the invention are administered in combination with a second therapeutic regimen.

The second therapeutic regimen is pomalidomide and dexamethasone administered at dosages recommended for the treatment of MM on a 28-day cycle.

The dosages of the second therapeutic regimen are pomalidomide administered at 4 mg/day on days 1-21 of each 28-day cycle and dexamethasone administered at 20 mg (for patients >75 years old) and 40 mg dosages weekly.

The pharmaceutical composition of the methods of the invention may be administered simultaneously or sequentially with the second therapeutic regimen.

The second therapeutic regimen may be administered orally with the pharmaceutical composition of the methods of the invention.

### Administration

The pharmaceutical compositions of the invention are administered as a fixed combination, e.g., as a unit dosage form (or dosage unit form). Fixed combinations may be advantageous for ease of administration and uniformity of dosage.

The pharmaceutical composition of the invention may be administered in a total volume of about 15 mL.

The pharmaceutical compositions of the invention may be administered once weekly for eight weeks, followed by once in two weeks for 16 weeks, followed by once in four weeks.

The pharmaceutical composition of the invention may be administered subcutaneously to the abdominal region.

Subcutaneous administration may be accomplished using a device. The device may be a syringe, a prefilled syringe, an auto-injector, either disposable or reusable, a pen injector, a patch injector, a wearable injector or an ambulatory syringe infusion pump with subcutaneous infusion sets.

While having described the invention in general terms, the embodiments of the invention will be further disclosed in the following examples that should not be construed as limiting the scope of the claims.

### Example 1: Fda-Approved Prescribing Information for Darzalex Faspro®

### Full Prescribing Information

### 1 INDICATIONS AND USAGE

### 1 Multiple Myeloma

DARZALEX FASPRO is indicated for the treatment of adult patients with multiple myeloma:
in combination with bortezomib, melphalan and prednisone in newly diagnosed patients who are ineligible for autologous stem cell transplant.
in combination with lenalidomide and dexamethasone in newly diagnosed patients who are ineligible for autologous stem cell transplant and in patients with relapsed or refractory multiple myeloma who have received at least one prior therapy.
in combination with bortezomib, thalidomide, and dexamethasone in newly diagnosed patients who are eligible for autologous stem cell transplant.
in combination with bortezomib and dexamethasone in patients who have received at least one prior therapy.
in combination with pomalidomide and dexamethasone in patients who have received at least one prior line of therapy including lenalidomide and a proteasome inhibitor.
as monotherapy, in patients who have received at least three prior lines of therapy including a proteasome inhibitor (PI) and an immunomodulatory agent or who are double-refractory to a PI and an immunomodulatory agent.

### 1.2 Light Chain Amyloidosis

DARZALEX FASPRO in combination with bortezomib, cyclophosphamide and dexamethasone is indicated for the treatment of adult patients with newly diagnosed light chain (AL) amyloidosis.

This indication is approved under accelerated approval based on response rate [see Clinical Studies (14.1)]. Continued approval for this indication may be contingent upon verification and description of clinical benefit in a confirmatory trial(s).

### Limitations of Use

DARZALEX FASPRO is not indicated and is not recommended for the treatment of patients with light chain (AL) amyloidosis who have NYHA Class IIIB or Class IV cardiac disease or Mayo Stage IIIB outside of controlled clinical trials [see Warnings andPrecautions (5.2)].

### 2 DOSAGE AND ADMINISTRATION

### 2.1 Important Dosing Information

DARZALEX FASPRO is for subcutaneous use only.
Administer medications before and after administration of DARZALEX FASPRO to minimize administration-related reactions [see Dosage and Administration (2.5)].
Type and screen patients prior to starting DARZALEX FASPRO.

### 2.2 Recommended Dosage for Multiple Myeloma

The recommended dose of DARZALEX FASPRO is 1,800 mg/30,000 units (1,800 mg daratumumab and 30,000 units hyaluronidase) administered subcutaneously over approximately 3-5 minutes. Tables 1, 2, 3, and 4 provide the recommended dosing schedule when DARZALEX FASPRO is administered as monotherapy or as part of a combination therapy.

Monotherapy and In Combination with Lenalidomide (DARZALEX FASPRO-Rd) or Pomalidomide (DARZALEX FASPRO-Pd) and Dexamethasone Use the dosing schedule provided in Table 1 when DARZALEX FASPRO is administered:
in combination with lenalidomide and dexamethasone (4-week cycle) OR
in combination with pomalidomide and dexamethasone (4-week cycle) OR
as monotherapy.

TABLE 1
DARZALEX FASPRO dosing schedule
in combination with lenalidomide or pomalidomide
and dexamethasone (4-week cycle) and for monotherapy

| Weeks | Schedule |
|---|---|
| Weeks 1 to 8 | weekly (total of 8 doses) |
| Weeks 9 to 24^{a} | every two weeks (total of 8 doses) |
| Week 25 onwards | every four weeks |
| until disease progression^{b} | |

^{a}First dose of the every-2-week dosing schedule is given at Week 9
^{b}First dose of the every-4-week dosing schedule is given at Week 25

When DARZALEX FASPRO is administered as part of a combination therapy, see Clinical Studies (14.2) and the prescribing information for dosage recommendations for the other drugs.

In Combination with Bortezomib, Melphalan and Prednisone (DARZALEX FASPRO-VMP)

Use the dosing schedule provided in Table 2 when DARZALEX FASPRO is administered in combination with bortezomib, melphalan and prednisone (6-week cycle).

TABLE 2
DARZALEX FASPRO dosing schedule in combination with
bortezomib, melphalan and prednisone (6-week cycle)

| Weeks | Schedule |
|---|---|
| Weeks 1 to 6 | weekly (total of 6 doses) |
| Weeks 7 to 54^{a} | every three weeks (total of 16 doses) |
| Week 55 onwards | every four weeks |
| until disease progression^{b} | |

^{a}First dose of the every-3-week dosing schedule is given at Week 7
^{b}First dose of the every-4-week dosing schedule is given at Week 55

When DARZALEX FASPRO is administered as part of a combination therapy, see Clinical Studies (14.1) and the prescribing information for dosage recommendations for the other drugs.

In Combination with Bortezomib, Thalidomide, and Dexamethasone (DARZALEX FASPRO-VTd)

Use the dosing schedule in Table 3 when DARZALEX FASPRO is administered in combination with bortezomib, thalidomide, and dexamethasone (4-week cycle).

TABLE 3
DARZALEX FASPRO dosing schedule in combination with
bortezomib, thalidomide and dexamethasone (4-week cycle)

| Treatment phase | Weeks | Schedule |
|---|---|---|
| Induction | Weeks 1 to 8 | weekly (total of 8 doses) |
| | Weeks 9 to 16^{a} | every two weeks (total of 4 doses) |

Stop for high dose chemotherapy and ASCT

| | | |
|---|---|---|
| Consolidation | Weeks 1 to 8^{b} | every two weeks (total of 4 doses) |

^{a}First dose of the every-2-week dosing schedule is given at Week 9
^{b}First dose of the every-2-week dosing schedule is given at Week 1 upon re-initiation of treatment following ASCT

When DARZALEX FASPRO is administered as part of a combination therapy, see the prescribing information for dosage recommendations for the other drugs.

In Combination with Bortezomib and Dexamethasone (DARZALEX FASPRO-Vd)

Use the dosing schedule in Table 4 when DARZALEX FASPRO is administered in combination with bortezomib and dexamethasone (3-week cycle).

TABLE 4
DARZALEX FASPRO dosing schedule in combination
with bortezomib and dexamethasone (3-week cycle)

| Weeks | Schedule |
|---|---|
| Weeks 1 to 9 | weekly (total of 9 doses) |
| Weeks 10 to 24^{a} | every three weeks (total of 5 doses) |
| Week 25 onwards | every four weeks |
| until disease progression^{b} | |

^{a}First dose of the every-3-week dosing schedule is given at Week 10
^{b}First dose of the every-4-week dosing schedule is given at Week 25

When DARZALEX FASPRO is administered as part of a combination therapy, see the prescribing information for dosage recommendations for the other drugs.

### 2.3 Recommended Dosage for Light Chain Amyloidosis

In Combination with Bortezomib, Cyclophosphamide and Dexamethasone (DARZALEX FASPRO-VCd)

Use the dosing schedule provided in Table 5 when DARZALEX FASPRO is administered in combination with bortezomib, cyclophosphamide and dexamethasone (4-week cycle).

TABLE 5
DARZALEX FASPRO dosing schedule in combination with
bortezomib, cyclophosphamide and dexamethasone (4-week cycle)

| Weeks | Schedule |
|---|---|
| Weeks 1 to 8 | weekly (total of 8 doses) |
| Weeks 9 to 24^{a} | every two weeks (total of 8 doses) |
| Week 25 onwards until | every four weeks |
| disease progression or a | |
| maximum of 2 years^{b} | |

^{a}First dose of the every-2-week dosing schedule is given at Week 9
^{b}First dose of the every-4-week dosing schedule is given at Week 25

When DARZALEX FASPRO is administered as part of a combination therapy, see Clinical Studies (14.2) and the prescribing information for dosage recommendations for the other drugs.

### 2.4 Administration

If a dose of DARZALEX FASPRO is missed, administer the dose as soon as possible and adjust the dosing schedule to maintain the dosing interval.

### 2.5 Recommended Concomitant Medications

### Pre-Medication

Administer the following pre-medications 1-3 hours before each dose of DARZALEX FASPRO:
Acetaminophen 650 to 1,000 mg orally
Diphenhydramine 25 to 50 mg (or equivalent) orally or intravenously
Corticosteroid (long- or intermediate-acting)

Monotherapy
Administer methylprednisolone 100 mg (or equivalent) orally or intravenously. Consider reducing the dose of methylprednisolone to 60 mg (or equivalent) following the second dose of DARZALEX FASPRO.

In Combination
Administer dexamethasone 20 mg (or equivalent) orally or intravenously prior to every DARZALEX FASPRO administration.
When dexamethasone is the background regimen-specific corticosteroid, the dexamethasone dose that is part of the background regimen will serve as pre-medication on DARZALEX FASPRO administration days [see Clinical Studies (14)].
Do not administer background regimen-specific corticosteroids (e.g. prednisone) on DARZALEX FASPRO administration days when patients have received dexamethasone (or equivalent) as a pre-medication.

### Post-Medication

Administer the following post-medications:

Monotherapy
Administer methylprednisolone 20 mg (or an equivalent dose of an intermediate- or long-acting corticosteroid) orally for 2 days starting the day after the administration of DARZALEX FASPRO.

In Combination
Consider administering oral methylprednisolone at a dose of less than or equal to 20 mg (or an equivalent dose of an intermediate- or long-acting corticosteroid) beginning the day after administration of DARZALEX FASPRO.
If a background regimen-specific corticosteroid (e.g. dexamethasone, prednisone) is administered the day after the administration of DARZALEX FASPRO, additional corticosteroids may not be needed [see Clinical Studies (14)].

If the patient does not experience a major systemic administration-related reaction after the first 3 doses of DARZALEX FASPRO, consider discontinuing the administration of corticosteroids (excluding any background regimen-specific corticosteroid).

For patients with a history of chronic obstructive pulmonary disease, consider prescribing short and long-acting bronchodilators and inhaled corticosteroids. Following the first 4 doses of DARZALEX FASPRO, consider discontinuing these additional post-medications, if the patient does not experience a major systemic administration-related reaction.

### Prophylaxis for Herpes Zoster Reactivation

Initiate antiviral prophylaxis to prevent herpes zoster reactivation within 1 week after starting DARZALEX FASPRO and continue for 3 months following the end of treatment [see Adverse Reactions (6.1)].

### 2.6 Dosage Modifications for Adverse Reactions

No dose reductions of DARZALEX FASPRO are recommended. Consider withholding DARZALEX FASPRO to allow recovery of blood cell counts in the event of myelosuppression [see Warnings and Precautions (5.3, 5.4)].

### 2.7 Preparation and Administration

DARZALEX FASPRO should be administered by a healthcare provider.

To prevent medication errors, check the vial labels to ensure that the drug being prepared and administered is DARZALEX FASPRO for subcutaneous use. Do not administer DARZALEX FASPRO intravenously.

DARZALEX FASPRO is ready to use.

### Preparation

Remove the DARZALEX FASPRO vial from refrigerated storage [2° C. to 8° C. (36° F. to 46° F.)] and equilibrate to ambient temperature [15° C. to 30° C. (59° F. to 86° F.)]. Store the unpunctured vial at ambient temperature and ambient light for a maximum of 24 hours. Keep out of direct sunlight. Do not shake.
Withdraw 15 mL from the vial into a syringe.
DARZALEX FASPRO is compatible with polypropylene or polyethylene syringe material; polypropylene, polyethylene, or polyvinyl chloride (PVC) subcutaneous infusion sets; and stainless steel transfer and injection needles. Use the product immediately.
After the solution of DARZALEX FASPRO is withdrawn into the syringe, replace the transfer needle with a syringe closing cap. Label the syringe appropriately to include the route of administration per institutional standards. Label the syringe with the peel-off label.
To avoid needle clogging, attach the hypodermic injection needle or subcutaneous infusion set to the syringe immediately prior to injection.
Parenteral drug products should be inspected visually for particulate matter and discoloration prior to administration, whenever solution and container permit. Do not use if opaque particles, discoloration or other foreign particles are present.

### Storage

If the syringe containing DARZALEX FASPRO is not used immediately, store the DARZALEX FASPRO solution for up to 4 hours at ambient temperature and ambient light. Discard after 4 hours, if not used.

### Administration

Inject 15 mL of DARZALEX FASPRO into the subcutaneous tissue of the abdomen approximately 3 inches [7.5 cm] to the right or left of the navel over approximately 3-5 minutes. No data are available on performing the injection at other sites of the body.
Rotate injection sites for successive injections.
Never inject DARZALEX FASPRO into areas where the skin is red, bruised, tender, hard or areas where there are scars.
Pause or slow down delivery rate if the patient experiences pain. In the event pain is not alleviated by pausing or slowing down delivery rate, a second injection site may be chosen on the opposite side of the abdomen to deliver the remainder of the dose.
During treatment with DARZALEX FASPRO, do not administer other medications for subcutaneous use at the same site as DARZALEX FASPRO.

### 3 DOSAGE FORMS AND STRENGTHS

Injection: 1,800 mg daratumumab and 30,000 units hyaluronidase per 15 mL (120 mg and 2,000 units/mL) colorless to yellow and clear to opalescent solution in a single-dose vial.

### 4 CONTRAINDICATIONS

DARZALEX FASPRO is contraindicated in patients with a history of severe hypersensitivity to daratumumab, hyaluronidase or any of the components of the formulation [see Warnings and Precautions (5.1) and Adverse Reactions (6.3)].

### 5 WARNINGS AND PRECAUTIONS

### 5.1 Hypersensitivity and Other Administration Reactions

Both systemic administration-related reactions, including severe or life-threatening reactions, and local injection-site reactions can occur with DARZALEX FASPRO. Fatal reactions have been reported with daratumumab-containing products, including DARZALEX FASPRO [see Adverse Reactions (6.3)].

### Systemic Reactions

In a pooled safety population of 832 patients with multiple myeloma (N=639) or light chain (AL) amyloidosis (N=193) who received DARZALEX FASPRO as monotherapy or as part of a combination therapy, 9% of patients experienced a systemic administration-related reaction (Grade 2: 3.5%, Grade 3: 0.8%). Systemic administration-related reactions occurred in 8% of patients with the first injection, 0.4% with the second injection, and cumulatively 1% with subsequent injections. The median time to onset was 3.2 hours (range: 9 minutes to 3.5 days). Of the 129 systemic administration-related reactions that occurred in 74 patients, 110 (85%) occurred on the day of DARZALEX FASPRO administration. Delayed systemic administration-related reactions have occurred in 1% of the patients.

Severe reactions included hypoxia, dyspnea, hypertension and tachycardia. Other signs and symptoms of systemic administration-related reactions may include respiratory symptoms, such as bronchospasm, nasal congestion, cough, throat irritation, allergic rhinitis, and wheezing, as well as anaphylactic reaction, pyrexia, chest pain, pruritus, chills, vomiting, nausea, and hypotension.

Pre-medicate patients with histamine-1 receptor antagonist, acetaminophen and corticosteroids [see Dosage and Administration (2.5)]. Monitor patients for systemic administration-related reactions, especially following the first and second injections. For anaphylactic reaction or life-threatening (Grade 4) administration-related reactions, immediately and permanently discontinue DARZALEX FASPRO. Consider administering corticosteroids and other medications after the administration of DARZALEX FASPRO depending on dosing regimen and medical history to minimize the risk of delayed (defined as occurring the day after administration) systemic administration-related reactions [see Dosage and Administration (2.5)].

### Local Reactions

In this pooled safety population, injection-site reactions occurred in 8% of patients, including Grade 2 reactions in 0.6%. The most frequent (>1%) injection-site reaction was injection site erythema. These local reactions occurred a median of 5.5 minutes (range: 0 minutes to 6.5 days) after starting administration of DARZALEX FASPRO. Monitor for local reactions and consider symptomatic management.

5.2 Cardiac Toxicity in Patients with Light Chain (AL) Amyloidosis

Serious or fatal cardiac adverse reactions occurred in patients with light chain (AL) amyloidosis who received DARZALEX FASPRO in combination with bortezomib, cyclophosphamide and dexamethasone [see Adverse Reactions (6.1)]. Serious cardiac disorders occurred in 16% and fatal cardiac disorders occurred in 10% of patients. Patients with NYHA Class IIIA or Mayo Stage IIIA disease may be at greater risk. Patients with NYHA Class IIIB or IV disease were not studied.

Monitor patients with cardiac involvement of light chain (AL) amyloidosis more frequently for cardiac adverse reactions and administer supportive care as appropriate.

### 5.3 Neutropenia

Daratumumab may increase neutropenia induced by background therapy [see Adverse Reactions (6.1)].

Monitor complete blood cell counts periodically during treatment according to manufacturer's prescribing information for background therapies. Monitor patients with neutropenia for signs of infection. Consider withholding DARZALEX FASPRO until recovery of neutrophils. In lower body weight patients receiving DARZALEX FASPRO, higher rates of Grade 3-4 neutropenia were observed.

### 5.4 Thrombocytopenia

Daratumumab may increase thrombocytopenia induced by background therapy [see Adverse Reactions (6.1)].

Monitor complete blood cell counts periodically during treatment according to manufacturer's prescribing information for background therapies. Consider withholding DARZALEX FASPRO until recovery of platelets.

### 5.5 Embryo-Fetal Toxicity

Based on the mechanism of action, DARZALEX FASPRO can cause fetal harm when administered to a pregnant woman. DARZALEX FASPRO may cause depletion of fetal immune cells and decreased bone density. Advise pregnant women of the potential risk to a fetus. Advise females with reproductive potential to use effective contraception during treatment with DARZALEX FASPRO and for 3 months after the last dose [see Use in Specific Populations (8.1, 8.3)].

The combination of DARZALEX FASPRO with lenalidomide, thalidomide or pomalidomide is contraindicated in pregnant women, because lenalidomide, thalidomide or pomalidomide may cause birth defects and death of the unborn child. Refer to the lenalidomide, thalidomide or pomalidomide prescribing information on use during pregnancy.

5.6 Interference with Serological Testing

Daratumumab binds to CD38 on red blood cells (RBCs) and results in a positive Indirect Antiglobulin Test (Indirect Coombs test). Daratumumab-mediated positive indirect antiglobulin test may persist for up to 6 months after the last daratumumab administration. Daratumumab bound to RBCs masks detection of antibodies to minor antigens in the patient's serum [see References (15)]. The determination of a patient's ABO and Rh blood type are not impacted [see Drug Interactions (7.1)].

Notify blood transfusion centers of this interference with serological testing and inform blood banks that a patient has received DARZALEX FASPRO. Type and screen patients prior to starting DARZALEX FASPRO [see Dosage and Administration (2.1)].

5.7 Interference with Determination of Complete Response

Daratumumab is a human IgG kappa monoclonal antibody that can be detected on both the serum protein electrophoresis (SPE) and immunofixation (IFE) assays used for the clinical monitoring of endogenous M-protein [see Drug Interactions (7.1)]. This interference can impact the determination of complete response and of disease progression in some DARZALEX FASPRO-treated patients with IgG kappa myeloma protein.

### 6 ADVERSE REACTIONS

The following clinically significant adverse reactions are described elsewhere in the labeling:
Hypersensitivity and Other Administration Reactions [see Warning and Precautions (5.1)].
Cardiac Toxicity in Patients with Light Chain (AL) Amyloidosis [see Warning and Precautions (5.2)].
Neutropenia [see Warning and Precautions (5.3)].
Thrombocytopenia [see Warning andPrecautions (5.4)].

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Newly Diagnosed Multiple Myeloma

In Combination with Bortezomib, Melphalan and Prednisone

The safety of DARZALEX FASPRO with bortezomib, melphalan and prednisone was evaluated in a single-arm cohort of PLEIADES [see Clinical Studies (14.1)]. Patients received DARZALEX FASPRO 1,800 mg/30,000 units administered subcutaneously once weekly from weeks 1 to 6, once every 3 weeks from weeks 7 to 54 and once every 4 weeks starting with week 55 until disease progression or unacceptable toxicity (N=67) in combination with bortezomib, melphalan and prednisone. Among these patients, 93% were exposed for 6 months or longer and 19% were exposed for greater than one year.

Serious adverse reactions occurred in 39% of patients who received DARZALEX FASPRO. Serious adverse reactions in >5% of patients included pneumonia and pyrexia. Fatal adverse reactions occurred in 3% of patients.

Permanent discontinuation of DARZALEX FASPRO due to an adverse reaction occurred in 4.5% of patients. The adverse reaction resulting in permanent discontinuation of DARZALEX FASPRO in more than 1 patient was neutropenic sepsis.

Dosage interruptions (defined as dose delays or skipped doses) due to an adverse reaction occurred in 51% of patients who received DARZALEX FASPRO. Adverse reactions requiring dosage interruptions in >5% of patients included thrombocytopenia, neutropenia, anemia, and pneumonia.

The most common adverse reactions (≥20%) were upper respiratory tract infection, constipation, nausea, fatigue, pyrexia, peripheral sensory neuropathy, diarrhea, cough, insomnia, vomiting, and back pain.

Table 6 summarizes the adverse reactions in patients who received DARZALEX FASPRO in PLEIADES.

TABLE 6
Adverse Reactions (≥10%) in Patients Who Received
DARZALEX FASPRO with Bortezomib, Melphalan and
Prednisone (DARZALEX FASPRO-VMP) in PLEIADES

| | DARZALEX FASPRO with | |
|---|---|---|
| | Bortezomib, Melphalan and Prednisone | |
| | (N = 67) | |
| | All Grades | Grades ≥ 3 |
| Adverse Reaction | (%) | (%) |

Infections

| | | |
|---|---|---|
| Upper respiratory tract infection^{a} | 39 | 0 |
| Bronchitis | 16 | 0 |
| Pneumonia^{b} | 15 | 7^{#} |

Gastrointestinal disorders

| | | |
|---|---|---|
| Constipation | 37 | 0 |
| Nausea | 36 | 0 |
| Diarrhea | 33 | 3^{#} |
| Vomiting | 21 | 0 |
| Abdominal pain^{c} | 13 | 0 |

General disorders and administration site conditions

| | | |
|---|---|---|
| Fatigue^{d} | 36 | 3 |
| Pyrexia | 34 | 0 |
| Edema peripheral^{e} | 13 | 1^{#} |

Nervous system disorders

| | | |
|---|---|---|
| Peripheral sensory neuropathy | 34 | 1^{#} |
| Dizziness | 10 | 0 |

Respiratory, thoracic and mediastinal disorders

| | | |
|---|---|---|
| Cough | 24 | 0 |

Psychiatric disorders

| | | |
|---|---|---|
| Insomnia | 22 | 3^{#} |

Musculoskeletal and connective tissue disorders

| | | |
|---|---|---|
| Back pain | 21 | 3^{#} |
| Musculoskeletal chest pain | 12 | 0 |

Metabolism and nutrition disorders

| | | |
|---|---|---|
| Decreased appetite | 15 | 1^{#} |

Skin and subcutaneous tissue disorders

| | | |
|---|---|---|
| Rash | 13 | 0 |
| Pruritus | 12 | 0 |

Vascular disorders

| | | |
|---|---|---|
| Hypertension | 13 | 6^{#} |
| Hypotension | 10 | 3^{#} |

^{a}Upper respiratory tract infection includes nasopharyngitis, respiratory syncytial virus infection, respiratory tract infection, rhinitis, tonsillitis, upper respiratory tract infection, and viral pharyngitis.
^{b}Pneumonia includes lower respiratory tract infection, lung infection, pneumocystis jirovecii pneumonia, pneumonia, and pneumonia bacterial.
^{c}Abdominal pain includes abdominal pain, and abdominal pain upper.
^{d}Fatigue includes asthenia, and fatigue.
^{e}Edema peripheral includes edema, edema peripheral, and peripheral swelling.
^{f}Cough includes cough, and productive cough.
^{#}Only grade 3 adverse reactions occurred.

Clinically relevant adverse reactions in <10% of patients who received DARZALEX FASPRO with bortezomib, melphalan and prednisone included:
General disorders and administration site conditions: infusion reaction, injection site reaction, chills
Infections: herpes zoster, urinary tract infection, influenza, sepsis
Musculoskeletal and connective tissue disorders: arthralgia, muscle spasms
Nervous system disorders: headache, paresthesia
Metabolism and nutrition disorders: hypocalcemia, hyperglycemia
Respiratory, thoracic and mediastinal disorders: dyspnea, pulmonary edema
Cardiac disorders: atrial fibrillation

Table 7 summarizes the laboratory abnormalities in patients who received DARZALEX FASPRO in PLEIADES.

TABLE 7
Select Hematology Laboratory Abnormalities Worsening from Baseline
in Patients Who Received DARZALEX FASPRO with Bortezomib,
Melphalan and Prednisone (DARZALEX FASPRO-VMP) in PLEIADES

| | DARZALEX FASPRO with | |
|---|---|---|
| | Bortezomib, Melphalan and | |
| | Prednisone^{a} | |
| | All Grades | Grades 3-4 |
| Laboratory Abnormality | (%) | (%) |
| Decreased leukocytes | 96 | 52 |
| Decreased lymphocytes | 93 | 84 |
| Decreased platelets | 93 | 42 |
| Decreased neutrophils | 88 | 49 |
| Decreased hemoglobin | 48 | 19 |

^{a}Denominator is based on the safety population treated with DARZALEX FASPRO-VMP (N = 67).

### Relapsed/Refractory Multiple Myeloma

In Combination with Lenalidomide and Dexamethasone

The safety of DARZALEX FASPRO with lenalidomide and dexamethasone was evaluated in a single-arm cohort of PLEIADES [see Clinical Studies (14.2)]. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity (N=65) in combination with lenalidomide and dexamethasone. Among these patients, 92% were exposed for 6 months or longer and 20% were exposed for greater than one year.

Serious adverse reactions occurred in 48% of patients who received DARZALEX FASPRO. Serious adverse reactions in >5% of patients included pneumonia, influenza and diarrhea. Fatal adverse reactions occurred in 3.1% of patients.

Permanent discontinuation of DARZALEX FASPRO due to an adverse reaction occurred in 11% of patients who received DARZALEX FASPRO. Adverse reactions resulting in permanent discontinuation of DARZALEX FASPRO in more than 1 patient were pneumonia and anemia.

Dosage interruptions due to an adverse reaction occurred in 6300 of patients who received DARZALEX FASPRO. Adverse reactions requiring dosage interruptions in >50 of patients included neutropenia, pneumonia, upper respiratory tract infection, influenza, dyspnea, and blood creatinine increased.

The most common adverse reactions (≥20%) were fatigue, diarrhea, upper respiratory tract infection, muscle spasms, constipation, pyrexia, pneumonia, and dyspnea.

Table 8 summarizes the adverse reactions in patients who received DARZALEX FASPRO in PLEIADES.

TABLE 8
Adverse Reactions (≥10%) in Patients Who Received DARZALEX
FASPRO with Lenalidomide and Dexamethasone (DARZALEX
FASPRO-Rd) in PLEIADES

| | DARZALEX FASPRO | |
|---|---|---|
| | with Lenalidomide and | |
| | Dexamethasone | |
| | (N = 65) | |
| | All Grades | Grades ≥3 |
| Adverse Reaction | (%) | (%) |

General disorders and administration site conditions

| | | |
|---|---|---|
| Fatigue^{a} | 52 | 5^{#} |
| Pyrexia | 23 | 2^{#} |
| Edema peripheral | 18 | 3^{#} |

Gastrointestinal disorders

| | | |
|---|---|---|
| Diarrhea | 45 | 5^{#} |
| Constipation | 26 | 2^{#} |
| Nausea | 12 | 0 |
| Vomiting | 11 | 0 |

Infections

| | | |
|---|---|---|
| Upper respiratory tract infection^{b} | 43 | 3^{#} |
| Pneumonia^{c} | 23 | 17 |
| Bronchitis^{d} | 14 | 2^{#} |
| Urinary tract infection | 11 | 0 |

Musculoskeletal and connective tissue disorders

| | | |
|---|---|---|
| Muscle spasms | 31 | 2^{#} |
| Back pain | 14 | 0 |

Respiratory, thoracic and mediastinal disorders

| | | |
|---|---|---|
| Dyspnea^{e} | 22 | 3 |
| Cough^{f} | 14 | 0 |

Nervous system disorders

| | | |
|---|---|---|
| Peripheral sensory neuropathy | 17 | 2^{#} |

Psychiatric disorders

| | | |
|---|---|---|
| Insomnia | 17 | 5^{#} |

Metabolism and nutrition disorders

| | | |
|---|---|---|
| Hyperglycemia | 12 | 9^{#} |
| Hypocalcemia | 11 | 0 |

^{a}Fatigue includes asthenia, and fatigue.
^{b}Upper respiratory tract infection includes nasopharyngitis, pharyngitis, respiratory tract infection viral, rhinitis, sinusitis, upper respiratory tract infection, and upper respiratory tract infection bacterial.
^{c}Pneumonia includes lower respiratory tract infection, lung infection, and pneumonia.
^{d}Bronchitis includes bronchitis, and bronchitis viral.
^{e}Dyspnea includes dyspnea, and dyspnea exertional.
^{f}Cough includes cough, and productive cough.
^{#}Only grade 3 adverse reactions occurred.

Clinically relevant adverse reactions in <10% of patients who received DARZALEX FASPRO with lenalidomide and dexamethasone included:
Musculoskeletal and connective tissue disorders: arthralgia, musculoskeletal chest pain
Nervous system disorders: dizziness, headache, paresthesia
Skin and subcutaneous tissue disorders: rash, pruritus
Gastrointestinal disorders: abdominal pain
Infections: influenza, sepsis, herpes zoster
Metabolism and nutrition disorders: decreased appetite
Cardiac disorders: atrial fibrillation
General disorders and administration site conditions: chills, infusion reaction, injection site reaction
Vascular disorders: hypotension, hypertension

Table 9 summarizes the laboratory abnormalities in patients who received DARZALEX FASPRO in PLEIADES.

TABLE 9
Select Hematology Laboratory Abnormalities Worsening from Baseline
in Patients Who Received DARZALEX FASPRO with Lenalidomide
and Dexamethasone (DARZALEX FASPRO-Rd) in PLEIADES

| | DARZALEX FASPRO with | |
|---|---|---|
| | Lenalidomide and Dexamethasone^{a} | |
| | All Grades | Grades 3-4 |
| Laboratory Abnormality | (%) | (%) |
| Decreased leukocytes | 94 | 34 |
| Decreased lymphocytes | 82 | 58 |
| Decreased platelets | 86 | 9 |
| Decreased neutrophils | 89 | 52 |
| Decreased hemoglobin | 45 | 8 |

^{a}Denominator is based on the safety population treated with DARZALEX FASPRO-Rd (N = 65).

### In Combination with Pomalidomide and Dexamethasone

The safety of DARZALEX FASPRO with pomalidomide and dexamethasone compared to pomalidomide and dexamethasone (Pd) in patients who had received at least one prior line of therapy with lenalidomide and a proteasome inhibitor (PI) was evaluated in APOLLO [see Clinical Studies (14.2)]. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity in combination with pomalidomide and dexamethasone (n=149) or pomalidomide and dexamethasone (n=150). Among patients receiving DARZALEX FASPRO-Pd, 71% were exposed for 6 months or longer and 50% were exposed for greater than one year.

Serious adverse reactions occurred in 50% of patients who received DARZALEX FASPRO-Pd. The most frequent serious adverse reactions in >5% of patients who received DARZALEX FASPRO-Pd were pneumonia (15%) and lower respiratory tract infection (12%). Fatal adverse reactions occurred in 7% of patients who received DARZALEX FASPRO-Pd.

Permanent treatment discontinuation due to an adverse reaction occurred in 2% of patients who received DARZALEX FASPRO-Pd. No adverse reactions resulting in permanent discontinuation occurred in more than 1 patient.

The most common adverse reactions (≥20%) were fatigue, pneumonia, upper respiratory tract infection, and diarrhea.

Table 10 summarizes the adverse reactions in patients who received DARZALEX FASPRO in APOLLO.

TABLE 10
Adverse Reactions Reported in ≥10% of Patients and With at Least a 5% Greater Frequency
in the DARZALEX FASPRO-Pd Arm in APOLLO

| | DARZALEX FASPRO-Pd | | | |
|---|---|---|---|---|
| | (N = 149) | | Pd (N = 150) | |
| | All Grades | Grades ≥3 | All Grades | Grades ≥3 |
| Adverse Reaction | (%) | (%) | (%) | (%) |

General disorders and administration site conditions

| | | | | |
|---|---|---|---|---|
| Fatigue | 46 | 13 | 39 | 5* |
| Pyrexia | 19 | 0 | 14 | 0 |
| Edema peripheral^{b} | 15 | 0 | 9 | 0 |

Infections

| | | | | |
|---|---|---|---|---|
| Pneumonia^{c} | 38 | 23^{@} | 27 | 17^{@} |
| Upper respiratory infection^{d} | 36 | 1* | 22 | 2* |

Gastrointestinal disorders

| | | | | |
|---|---|---|---|---|
| Diarrhea | 22 | 54 | 14 | 1* |

Respiratory, thoracic and mediastinal disorders

| | | | | |
|---|---|---|---|---|
| Cough^{e} | 13 | 0 | 8 | 0 |

Key: Pd = pomalidomide-dexamethasone
^{a}Fatigue includes asthenia, and fatigue.
^{b}Edema peripheral includes edema, edema peripheral and peripheral swelling.
^{c}Pneumonia includes atypical pneumonia, lower respiratory tract infection, pneumonia, pneumonia aspiration, pneumonia bacterial, and pneumonia respiratory syncytial viral.
^{d}Upper respiratory tract infection includes nasopharyngitis, pharyngitis, respiratory syncytial virus infection, respiratory tract infection, respiratory tract infection viral, rhinitis, sinusitis, tonsillitis, upper respiratory tract infection, and viral upper respiratory tract infection.
^{e}Cough includes cough, and productive cough.
^{#}Only grade 3 adverse reactions occurred.
^{@}Grade 5 adverse reactions occurred, n = 3 (2.0%) in the DARZALEX FASPRO-Pd arm and n = 2 (1.3%) in the Pd arm.

Clinically relevant adverse reactions in <10% of patients who received DARZALEX FASPRO with pomalidomide and dexamethasone include:
Metabolism and nutrition disorders: hypocalcemia, hypokalemia, decreased appetite, dehydration
Nervous system disorders: peripheral sensory neuropathy, syncope, headache, paresthesia, dizziness
Musculoskeletal and connective tissue disorders: muscle spasms, musculoskeletal chest pain, arthralgia
Psychiatric disorders: insomnia
Gastrointestinal disorders: nausea, abdominal pain, vomiting
Skin and subcutaneous tissue disorders: rash, pruritus
Cardiac disorders: atrial fibrillation
General disorders and administration site conditions: infusion reactions, chills, injection site reaction
Infections: urinary tract infection, influenza, hepatitis B reactivation, herpes zoster, sepsis
Vascular disorders: hypertension, hypotension

Table 11 summarizes the laboratory abnormalities in patients who received DARZALEX FASPRO in APOLLO.

TABLE 11
Select Hematology Laboratory Abnormalities Worsening from Baseline in Patients Who
Received DARZALEX FASPRO-Pd or Pd in APOLLO

| | DARZALEX FASPRO-Pd^{a} | | Pd^{a} | |
|---|---|---|---|---|
| | All Grades | Grades 3-4 | All Grades | Grades 3-4 |
| Laboratory Abnormality | (%) | (%) | (%) | (%) |
| Decreased neutrophils | 97 | 84 | 84 | 63 |
| Decreased leukocytes | 95 | 64 | 82 | 40 |
| Decreased lymphocytes | 93 | 59 | 79 | 33 |
| Decreased platelets | 75 | 19 | 60 | 19 |
| Decreased hemoglobin | 51 | 16 | 57 | 15 |

Key: Pd = pomalidomide-dexamethasone
^{a}Denominator is based on number of subjects with a baseline and post-baseline laboratory value for each laboratory test: N = 148 for DARZALEX FASPRO-Pd and N = 149 for Pd.

### Monotherapy

The safety of DARZALEX FASPRO as monotherapy was evaluated in COLUMBA [see Clinical Trials (14.2)]. Patients received DARZALEX FASPRO 1,800 mg/30,000 units administered subcutaneously or daratumumab 16 mg/kg administered intravenously; each administered once weekly from weeks 1 to 8, once every 2 weeks from weeks 9 to 24 and once every 4 weeks starting with week 25 until disease progression or unacceptable toxicity. Among patients receiving DARZALEX FASPRO, 37% were exposed for 6 months or longer and 1% were exposed for greater than one year.

Serious adverse reactions occurred in 26% of patients who received DARZALEX FASPRO. Fatal adverse reactions occurred in 5% of patients. Fatal adverse reactions occurring in more than 1 patient were general physical health deterioration, septic shock, and respiratory failure.

Permanent discontinuation due to an adverse reaction occurred in 10% of patients who received DARZALEX FASPRO. Adverse reactions resulting in permanent discontinuation of DARZALEX FASPRO in more than 2 patients were thrombocytopenia and hypercalcemia.

Dosage interruptions due to an adverse reaction occurred in 26% of patients who received DARZALEX FASPRO. Adverse reactions requiring dosage interruption in >5% of patients included thrombocytopenia.

The most common adverse reaction (≥20%) was upper respiratory tract infection.

Table 12 summarizes the adverse reactions in COLUMBA.

TABLE 12
Adverse Reactions (≥10%) in Patients Who Received DARZALEX FASPRO or Intravenous
Daratumumab in COLUMBA

| | DARZALEX FASPRO | | Intravenous Daratumumab | |
|---|---|---|---|---|
| | (N = 260) | | (N = 258) | |
| | All Grades | Grade ≥3 | All Grades | Grade ≥3 |
| Adverse Reaction | (%) | (%) | (%) | (%) |

Infections

| | | | | |
|---|---|---|---|---|
| Upper respiratory tract infection^{a} | 24 | 1^{#} | 22 | 1^{#} |
| Pneumonia^{b} | 8 | 5 | 10 | 6^{@} |

Gastrointestinal disorders

| | | | | |
|---|---|---|---|---|
| Diarrhea | 15 | 1^{#} | 11 | 0.4^{#} |
| Nausea | 8 | 0.4^{#} | 11 | 0.4^{#} |

General disorders and administration site conditions

| | | | | |
|---|---|---|---|---|
| Fatigue^{c} | 15 | 1^{#} | 16 | 2^{#} |
| Infusion reactions^{d} | 13 | 2^{#} | 34 | 5^{#} |
| Pyrexia | 13 | 0 | 13 | 1^{#} |
| Chills | 6 | 0.4^{#} | 12 | 1^{#} |

Musculoskeletal and connective tissue disorders

| | | | | |
|---|---|---|---|---|
| Back pain | 10 | 2^{#} | 12 | 3^{#} |

Respiratory, thoracic and mediastinal disorders

| | | | | |
|---|---|---|---|---|
| Cough^{e} | 9 | 1^{#} | 14 | 0 |
| Dyspnea^{f} | 6 | 1^{#} | 11 | 1^{#} |

^{a}Upper respiratory tract infection includes acute sinusitis, nasopharyngitis, pharyngitis, respiratory syncytial virus infection, respiratory tract infection, rhinitis, rhinovirus infection, sinusitis, and upper respiratory tract infection.
^{b}Pneumonia includes lower respiratory tract infection, lung infection, pneumocystis jirovecii pneumonia, and pneumonia.
^{c}Fatigue includes asthenia, and fatigue.
^{d}Infusion reactions includes terms determined by investigators to be related to infusion.
^{e}Cough includes cough, and productive cough.
^{f}Dyspnea includes dyspnea, and dyspnea exertional.
^{#}Only grade 3 adverse reactions occurred.
^{@}Grade 5 adverse reactions occurred.

Clinically relevant adverse reactions in <10% of patients who received DARZALEX FASPRO included:
General disorders and administration site conditions: injection site reaction, peripheral edema
Musculoskeletal and connective tissue disorders: arthralgia, musculoskeletal chest pain, muscle spasms
Gastrointestinal disorders: constipation, vomiting, abdominal pain
Metabolism and nutrition disorders: decreased appetite, hyperglycemia, hypocalcemia, dehydration
Psychiatric disorders: insomnia
Vascular disorders: hypertension, hypotension
Nervous system disorders: dizziness, peripheral sensory neuropathy, paresthesia
Infections: bronchitis, influenza, urinary tract infection, herpes zoster, sepsis, hepatitis B virus reactivation
Skin and subcutaneous tissue disorders: pruritus, rash
Cardiac disorders: atrial fibrillation
Respiratory, thoracic and mediastinal disorders: pulmonary edema

Table 13 summarizes the laboratory abnormalities in COLUMBA.

TABLE 13
Select Hematology Laboratory Abnormalities Worsening from Baseline in Patients Receiving
DARZALEX FASPRO or Intravenous Daratumumab in COLUMBA

| | DARZALEX FASPRO^{a} | | Intravenous Daratumumab^{a} | |
|---|---|---|---|---|
| | All Grades | Grades 3-4 | All Grades | Grades 3-4 |
| Laboratory Abnormality | (%) | (%) | (%) | (%) |
| Decreased leukocytes | 65 | 19 | 57 | 14 |
| Decreased lymphocytes | 59 | 36 | 56 | 36 |
| Decreased neutrophils | 55 | 19 | 43 | 11 |
| Decreased platelets | 43 | 16 | 45 | 14 |
| Decreased hemoglobin | 42 | 14 | 39 | 16 |

^{a}Denominator is based on the safety population treated with DARZALEX FASPRO (N = 260) and Intravenous Daratumumab (N = 258).

### Light Chain Amyloidosis

### In Combination with Bortezomib, Cyclophosphamide and Dexamethasone

The safety of DARZALEX FASPRO with bortezomib, cyclophosphamide and dexamethasone (DARZALEX FASPRO-VCd) was evaluated in ANDROMEDA [see Clinical Studies (14.2)]. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity or a maximum of 2 years. Among patients who received DARZALEX FASPRO-VCd, 74% were exposed for 6 months or longer and 32% were exposed for greater than one year.

Serious adverse reactions occurred in 43% of patients who received DARZALEX FASPRO in combination with VCd. Serious adverse reactions that occurred in at least 5% of patients in the DARZALEX FASPRO-VCd arm were pneumonia (9%), cardiac failure (8%), and sepsis (5%). Fatal adverse reactions occurred in 11% of patients. Fatal adverse reactions that occurred in more than one patient included cardiac arrest (4%), sudden death (3%), cardiac failure (3%), and sepsis (1%).

Permanent discontinuation of DARZALEX FASPRO due to an adverse reaction occurred in 5% of patients. Adverse reactions resulting in permanent discontinuation of DARZALEX FASPRO in more than one patient were pneumonia, sepsis, and cardiac failure.

Dosage interruptions (defined as dose delays or skipped doses) due to an adverse reaction occurred in 36% of patients who received DARZALEX FASPRO. Adverse reactions which required a dosage interruption in ≥3% of patients included upper respiratory tract infection (9%), pneumonia (6%), cardiac failure (4%), fatigue (3%), herpes zoster (3%), dyspnea (3%), and neutropenia (3%).

The most common adverse reactions (≥20%) were upper respiratory tract infection, diarrhea, peripheral edema, constipation, fatigue, peripheral sensory neuropathy, nausea, insomnia, dyspnea, and cough.

Table 14 below summarizes the adverse reactions in patients who received DARZALEX FASPRO in ANDROMEDA.

TABLE 14
Adverse Reactions (≥10%) in Patients with AL Amyloidosis Who Received DARZALEX
FASPRO with Bortezomib, Cyclophosphamide and Dexamethasone (DARZALEX FASPRO-
VCd) with a Difference Between Arms of >5% Compared to VCd in ANDROMEDA

| | DARZALEX FASPRO-VCd | | VCd | |
|---|---|---|---|---|
| | (N = 193) | | (N = 188) | |
| | All Grades | Grades 3-4 | All Grades | Grades 3-4 |
| Adverse Reaction | (%) | (%) | (%) | (%) |

Infections

| | | | | |
|---|---|---|---|---|
| Upper respiratory tract infection^{a} | 40 | 1^{#} | 21 | 1^{#} |
| Pneumonia^{b} | 15 | 10 | 9 | 5 |

Gastrointestinal disorders

| | | | | |
|---|---|---|---|---|
| Diarrhea | 36 | 6^{#} | 30 | 4 |
| Constipation | 34 | 2^{#} | 29 | 0 |

Nervous system disorders

| | | | | |
|---|---|---|---|---|
| Peripheral sensory neuropathy | 31 | 3^{#} | 20 | 2^{#} |

Respiratory, thoracic and mediastinal disorders

| | | | | |
|---|---|---|---|---|
| Dyspnea^{c} | 26 | 4 | 20 | 4^{#} |
| Cough^{d} | 20 | 1^{#} | 11 | 0 |

Musculoskeletal and connective tissue disorders

| | | | | |
|---|---|---|---|---|
| Back pain | 12 | 2^{#} | 6 | 0 |
| Arthralgia | 10 | 0 | 5 | 0 |
| Muscle spasms | 10 | 1^{#} | 5 | 0 |

Cardiac disorders

| | | | | |
|---|---|---|---|---|
| Arrhythmia^{e} | 11 | 4 | 5 | 2 |

General disorders and administration site conditions

| | | | | |
|---|---|---|---|---|
| Injection site reactions^{f} | 11 | 0 | 0 | 0 |

^{#}Only grade 3 adverse reactions occurred.
^{a}Upper respiratory tract infection includes laryngitis, nasopharyngitis, pharyngitis, respiratory syncytial virus infection, respiratory tract infection, respiratory tract infection viral, rhinitis, rhinovirus infection, sinusitis, tonsillitis, tracheitis, upper respiratory tract infection, upper respiratory tract infection bacterial, and viral upper respiratory tract infection.
^{b}Pneumonia includes lower respiratory tract infection, pneumonia, pneumonia aspiration, and pneumonia pneumococcal.
^{c}Dyspnea includes dyspnea, and dyspnea exertional.
^{d}Cough includes cough, and productive cough.
^{e}Arrhythmia includes atrial flutter, atrial fibrillation, supraventricular tachycardia, bradycardia, arrhythmia, bradyarrhythmia, cardiac flutter, extrasystoles, supraventricular extrasystoles, ventricular arrhythmia, ventricular extrasystoles, atrial tachycardia, ventricular tachycardia
^{f}Injection site reactions includes terms determined by investigators to be related to daratumumab injection.

Clinically relevant adverse reactions not included in Table 14 and occurred in patients who received DARZALEX FASPRO with bortezomib, cyclophosphamide and dexamethasone included:
Skin and subcutaneous tissue disorders: rash, pruritus
Nervous system disorders: paresthesia
General disorders and administration site conditions: infusion reaction, chills
Cardiac disorders: cardiac failure^{a}, cardiac arrest ^{a} Cardiac failure includes cardiac dysfunction, cardiac failure, cardiac failure congestive, cardiovascular insufficiency, diastolic dysfunction, pulmonary edema, and left ventricular dysfunction occurred in 11% of patients.
Metabolism and nutrition disorders: hyperglycemia, hypocalcemia, dehydration
Infections: bronchitis, herpes zoster, sepsis, urinary tract infection, influenza
Vascular disorders: hypertension
Musculoskeletal and connective tissue disorders: musculoskeletal chest pain
Gastrointestinal disorders: pancreatitis
Respiratory, thoracic and mediastinal disorders: pulmonary edema

Table 15 summarizes the laboratory abnormalities in patients who received DARZALEX FASPRO in ANDROMEDA.

TABLE 15
Select Hematology Laboratory Abnormalities Worsening from Baseline in Patients Who
Received DARZALEX FASPRO with Bortezomib, Cyclophosphamide and
Dexamethasone (DARZALEX FASPRO-VCd) in ANDROMEDA

| | |
|---|---|
| DARZALEX FASPRO-VCd | VCd |

| | All Grades | Grades 3-4 | All Grades | Grades 3-4 |
|---|---|---|---|---|
| Laboratory Abnormality | (%) | (%) | (%) | (%) |
| Decreased lymphocytes | 81 | 54 | 71 | 46 |
| Decreased hemoglobin | 66 | 6 | 70 | 6 |
| Decreased leukocytes | 60 | 7 | 46 | 4 |
| Decreased platelets | 46 | 3 | 40 | 4 |
| Decreased neutrophils | 30 | 6 | 18 | 4 |

Denominator is based on the number of patients with a baseline and post-baseline laboratory value for each laboratory test, N = 188 for DARZALEX FASPRO-VCd and N = 186 for VCd.

### Cardiac Adverse Reactions in Light Chain (AL) Amyloidosis

Among patients who received DARZALEX FASPRO in combination with VCd, 72% of patients had baseline cardiac involvement with Mayo Cardiac Stage I (3%), Stage 11 (46%) and Stage III (51%). Serious cardiac disorders occurred in 16% of patients (8% of patients with Mayo Cardiac Stage I and II and 28% of patients with Stage III). Serious cardiac disorders in >2% of patients included cardiac failure (8%), cardiac arrest (4%) and arrhythmia (4%). Fatal cardiac disorders occurred in 10% of patients (5% of patients with Mayo Cardiac Stage I and II and 19% of patients with Stage III) who received DARZALEX FASPRO in combination with VCd. Fatal cardiac disorders that occurred in more than one patient in the DARZALEX FASPRO-VCd arm included cardiac arrest (4%), sudden death (3%), and cardiac failure (3%).

### 6.2 Immunogenicity

As with all therapeutic proteins, there is the potential for immunogenicity. The detection of antibody formation is highly dependent on the sensitivity and specificity of the assay. Additionally, the observed incidence of antibody (including neutralizing antibody) positivity in an assay may be influenced by several factors including assay methodology, sample handling, timing of sample collection, concomitant medications, and underlying disease. For these reasons, comparison of the incidence of antibodies in the studies described below with the incidence of antibodies in other studies or to other daratumumab products or other hyaluronidase products may be misleading.

In patients with multiple myeloma and light chain (AL) amyloidosis who received DARZALEX FASPRO as monotherapy or as part of a combination therapy, less than 1% of 756 patients developed treatment-emergent anti-daratumumab antibodies.

In patients with multiple myeloma and light chain (AL) amyloidosis who received DARZALEX FASPRO as monotherapy or as part of a combination therapy, 7% of 750 patients developed treatment-emergent anti-rHuPH20 antibodies. The anti-rHuPH20 antibodies did not appear to affect daratumumab exposure. None of the patients who tested positive for anti-rHuPH20 antibodies tested positive for neutralizing antibodies.

### 6.3 Postmarketing Experience

The following adverse reactions have been identified with post-approval use of daratumumab. Because these reactions are reported voluntarily from a population of uncertain size, it is not always possible to reliably estimate their frequency or establish a causal relationship to drug exposure.

Immune System: Anaphylactic reaction; systemic administration reactions (including death)

### Gastrointestinal: Pancreatitis

### Infections: Cytomegalovirus, Listeriosis

### 7 DRUG INTERACTIONS

### 7.1 Effects of Daratumumab on Laboratory Tests

Interference with Indirect Antiglobulin Tests (Indirect Coombs Test)

Daratumumab binds to CD38 on RBCs and interferes with compatibility testing, including antibody screening and cross matching. Daratumumab interference mitigation methods include treating reagent RBCs with dithiothreitol (DTT) to disrupt daratumumab binding [see References (15)] or genotyping. Since the Kell blood group system is also sensitive to DTT treatment, supply K-negative units after ruling out or identifying alloantibodies using DTT-treated RBCs.

If an emergency transfusion is required, administer non-cross-matched ABO/RhD-compatible RBCs per local blood bank practices.

Interference with Serum Protein Electrophoresis and Immunofixation Tests

Daratumumab may be detected on serum protein electrophoresis (SPE) and immunofixation (IFE) assays used for monitoring disease monoclonal immunoglobulins (M protein). False positive SPE and IFE assay results may occur for patients with IgG kappa myeloma protein impacting initial assessment of complete responses by International Myeloma Working Group (IMWG) criteria. In DARZALEX FASPRO-treated patients with persistent very good partial response, where daratumumab interference is suspected, consider using a FDA-approved daratumumab-specific IFE assay to distinguish daratumumab from any remaining endogenous M protein in the patient's serum, to facilitate determination of a complete response.

### 8 USE IN SPECIFIC POPULATIONS

### 8.1 Pregnancy

### Risk Summary

DARZALEX FASPRO can cause fetal harm when administered to a pregnant woman. The assessment of associated risks with daratumumab products is based on the mechanism of action and data from target antigen CD38 knockout animal models (see Data). There are no available data on the use of DARZALEX FASPRO in pregnant women to evaluate drug-associated risk of major birth defects, miscarriage or adverse maternal or fetal outcomes. Animal reproduction studies have not been conducted.

The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2% to 4% and 15% to 20%, respectively.

The combination of DARZALEX FASPRO and lenalidomide, thalidomide or pomalidomide is contraindicated in pregnant women, because lenalidomide, thalidomide and pomalidomide may cause birth defects and death of the unborn child. Lenalidomide, thalidomide and pomalidomide are only available through a REMS program. Refer to the lenalidomide, thalidomide or pomalidomide prescribing information on use during pregnancy.

### Clinical Considerations

### Fetal/Neonatal Adverse Reactions

Immunoglobulin G1 (IgG1) monoclonal antibodies are transferred across the placenta. Based on its mechanism of action, DARZALEX FASPRO may cause depletion of fetal CD38 positive immune cells and decreased bone density. Defer administering live vaccines to neonates and infants exposed to daratumumab in utero until a hematology evaluation is completed.

### Data

### Animal Data

DARZALEX FASPRO for subcutaneous injection contains daratumumab and hyaluronidase. Mice that were genetically modified to eliminate all CD38 expression (CD38 knockout mice) had reduced bone density at birth that recovered by 5 months of age. Data from studies using CD38 knockout animal models also suggest the involvement of CD38 in the regulation of humoral immune responses (mice), feto-maternal immune tolerance (mice), and early embryonic development (frogs).

No systemic exposure of hyaluronidase was detected in monkeys given 22,000 U/kg subcutaneously (12 times higher than the human dose) and there were no effects on embryo-fetal development in pregnant mice given 330,000 U/kg hyaluronidase subcutaneously daily during organogenesis, which is 45 times higher than the human dose.

There were no effects on pre- and post-natal development through sexual maturity in offspring of mice treated daily from implantation through lactation with 990,000 U/kg hyaluronidase subcutaneously, which is 134 times higher than the human doses.

### 8.2 Lactation

### Risk Summary

There is no data on the presence of daratumumab and hyaluronidase in human milk, the effects on the breastfed child, or the effects on milk production. Maternal immunoglobulin G is known to be present in human milk. Published data suggest that antibodies in breast milk do not enter the neonatal and infant circulations in substantial amounts. Because of the potential for serious adverse reactions in the breastfed child when DARZALEX FASPRO is administered with lenalidomide, thalidomide or pomalidomide, advise women not to breastfeed during treatment with DARZALEX FASPRO. Refer to lenalidomide, thalidomide or pomalidomide prescribing information for additional information.

### Data

### Animal Data

No systemic exposure of hyaluronidase was detected in monkeys given 22,000 U/kg subcutaneously (12 times higher than the human dose) and there were no effects on post-natal development through sexual maturity in offspring of mice treated daily during lactation with 990,000 U/kg hyaluronidase subcutaneously, which is 134 times higher than the human doses.

### 8.3 Females and Males of Reproductive Potential

DARZALEX FASPRO can cause fetal harm when administered to a pregnant woman [see Use in Specific Populations (8.1)].

### Pregnancy Testing

With the combination of DARZALEX FASPRO with lenalidomide, thalidomide or pomalidomide, refer to the lenalidomide, thalidomide or pomalidomide labeling for pregnancy testing requirements prior to initiating treatment in females of reproductive potential.

### Contraception

Advise females of reproductive potential to use effective contraception during treatment with DARZALEX FASPRO and for 3 months after the last dose. Additionally, refer to the lenalidomide, thalidomide or pomalidomide labeling for additional recommendations for contraception.

### 8.4 Pediatric Use

Safety and effectiveness of DARZALEX FASPRO in pediatric patients have not been established.

### 8.5 Geriatric Use

Of the 291 patients who received DARZALEX FASPRO as monotherapy for relapsed and refractory multiple myeloma, 37% were 65 to <75 years of age, and 19% were 75 years of age or older. No overall differences in effectiveness of DARZALEX FASPRO have been observed between patients ≥65 years of age and younger patients. Adverse reactions that occurred at a higher frequency (≥5% difference) in patients ≥65 years of age included upper respiratory tract infection, urinary tract infection, dizziness, cough, dyspnea, diarrhea, nausea, fatigue, and peripheral edema. Serious adverse reactions that occurred at a higher frequency (≥2% difference) in patients ≥65 years of age included pneumonia.

Of the 214 patients who received DARZALEX FASPRO as combination therapy with pomalidomide and dexamethasone or DARZALEX FASPRO as combination therapy with lenalidomide and low-dose dexamethasone for relapsed and refractory multiple myeloma, 43% were 65 to <75 years of age, and 18% were 75 years of age or older. No overall differences in effectiveness were observed between patients ≥65 years (n=131) and <65 years (n=85). Adverse reactions occurring at a higher frequency (≥5% difference) in patients ≥65 years of age included fatigue, pyrexia, peripheral edema, urinary tract infection, diarrhea, constipation, vomiting, dyspnea, cough, and hyperglycemia. Serious adverse reactions occurring at a higher frequency (≥2% difference) in patients ≥65 years of age included neutropenia, thrombocytopenia, diarrhea, anemia, COVID-19, ischemic colitis, deep vein thrombosis, general physical health deterioration, pulmonary embolism, and urinary tract infection.

Of the 193 patients who received DARZALEX FASPRO as part of a combination therapy for light chain (AL) amyloidosis, 35% were 65 to <75 years of age, and 10% were 75 years of age or older. Clinical studies of DARZALEX FASPRO as part of a combination therapy for patients with light chain (AL) amyloidosis did not include sufficient numbers of patients aged 65 and older to determine whether effectiveness differs from that of younger patients. Adverse reactions that occurred at a higher frequency in patients 65 years of age were peripheral edema, asthenia, pneumonia and hypotension.

No clinically meaningful differences in the pharmacokinetics of daratumumab were observed in geriatric patients compared to younger adult patients [see ClinicalPharmacology (12.3)].

### 11 DESCRIPTION

Daratumumab is an immunoglobulin G1 kappa (IgG1κ) human monoclonal antibody that binds to the CD38 antigen. Daratumumab is produced in Chinese Hamster Ovary (CHO) cells using recombinant DNA technology. The molecular weight of daratumumab is approximately 148 kDa.

Hyaluronidase (recombinant human) is an endoglycosidase used to increase the dispersion and absorption of co-administered drugs when administered subcutaneously. It is a glycosylated single-chain protein produced by Chinese Hamster Ovary cells containing a DNA plasmid encoding for a soluble fragment of human hyaluronidase (PH20). Hyaluronidase (recombinant human) has a molecular weight of approximately 61 kDa.

DARZALEX FASPRO® (daratumumab and hyaluronidase-fihj) injection is a sterile, preservative-free, colorless to yellow, and clear to opalescent solution supplied in a single-dose vial for subcutaneous administration.

Each DARZALEX FASPRO 15 mL single-dose vial contains 1,800 mg of daratumumab and 30,000 units of hyaluronidase, L-histidine (4.9 mg), L-histidine hydrochloride monohydrate (18.4 mg), L-methionine (13.5 mg), polysorbate 20 (6 mg), sorbitol (735.1 mg), and Water for Injection, USP.

### 12 CLINICAL PHARMACOLOGY

### 12.1 Mechanism of Action

CD38 is a transmembrane glycoprotein (48 kDa) expressed on the surface of hematopoietic cells, including clonal plasma cells in multiple myeloma and light chain (AL) amyloidosis, as well as other cell types. Surface CD38 has multiple functions, including receptor mediated adhesion, signaling, and modulation of cyclase and hydrolase activity. Daratumumab is an IgG1κ human monoclonal antibody (mAb) that binds to CD38 and inhibits the growth of CD38 expressing tumor cells by inducing apoptosis directly through Fc mediated cross linking as well as by immune-mediated tumor cell lysis through complement dependent cytotoxicity (CDC), antibody dependent cell mediated cytotoxicity (ADCC) and antibody dependent cellular phagocytosis (ADCP). A subset of myeloid derived suppressor cells (CD38+MDSCs), regulatory T cells (CD38+T_{regs}) and B cells (CD38+B_{regs}) are decreased by daratumumab.

Hyaluronan is a polysaccharide found in the extracellular matrix of the subcutaneous tissue. It is depolymerized by the naturally occurring enzyme hyaluronidase. Unlike the stable structural components of the interstitial matrix, hyaluronan has a half-life of approximately 0.5 days. Hyaluronidase increases permeability of the subcutaneous tissue by depolymerizing hyaluronan. In the doses administered, hyaluronidase in DARZALEX FASPRO acts locally. The effects of hyaluronidase are reversible and permeability of the subcutaneous tissue is restored within 24 to 48 hours.

### 12.2 Pharmacodynamics

NK cells express CD38 and are susceptible to daratumumab mediated cell lysis. Decreases in absolute counts and percentages of total NK cells (CD16+CD56+) and activated (CD16+CD56^{dim}) NK cells in peripheral whole blood and bone marrow were observed with DARZALEX FASPRO treatment.

### Cardiac Electrophysiology

DARZALEX FASPRO as a large protein has a low likelihood of direct ion channel interactions. There is no evidence from non-clinical or clinical data to suggest that DARZALEX FASPRO has the potential to delay ventricular repolarization.

### Exposure-Response Relationship

The exposure-response relationship and time course of pharmacodynamics of DARZALEX FASPRO have not been fully characterized.

### 12.3 Pharmacokinetics

Following the recommended dose of DARZALEX FASPRO 1,800 mg/30,000 units subcutaneously once weekly for 8 weeks, daratumumab peak concentration (Cₘₐₓ) increased 4.8-fold and area under the curve (AUC_{0-7 days}) increased 5.4-fold from the 1^{st} dose to the 8^{th} dose as monotherapy. Maximum trough concentrations for DARZALEX FASPRO are typically observed at the end of the weekly dosing regimens for both monotherapy and combination therapies. The mean±standard deviation (SD) maximum trough serum concentration (C_{trough}) after the 8^{th} dose was 593±306 μg/mL when DARZALEX FASPRO was administered as monotherapy and 537±277 μg/mL and 526±226 μg/mL when DARZALEX FASPRO was administered as combination with Pd and Rd, respectively.

Table 16 lists the observed mean (±SD) maximum trough concentrations (C_{trough}) after the 8^{th} dose, simulated median (5^{th}-95^{th} percentiles) maximum C_{trough} after the 8^{th} dose, simulated median (5^{th}-95^{th} percentiles) Cₘₐₓ after the 8^{th} dose, and simulated median (5^{th}-95^{th} percentiles) area under the curve (AUC_{0-7 day}) after the 8^{th} dose following DARZALEX FASPRO 1,800 mg/30,000 units administered subcutaneously or daratumumab 16 mg/kg administered intravenously in patients with multiple myeloma or light chain (AL) amyloidosis.

TABLE 16
Daratumumab Exposure for Patients with Multiple Myeloma or Light Chain (AL)
Amyloidosis

| | Intravenous | | DARZALEX FASPRO | | DARZALEX FASPRO | |
|---|---|---|---|---|---|---|
| | Daratumumab | | 1,800 mg/30,000 units in | | 1,800 mg/30,000 units in | |
| | 16 mg/kg in Patients | | Patients with Multiple | | Patients with Light Chain | |
| Parameter | with Multiple Myeloma | | Myeloma | | (AL) Amyloidosis | |
| Observed mean ± SD | 522 ± 226^{a,b} | | 593 ± 306^{a,b} | | 597 ± 232^{c} | |
| max C_{trough} after 8^{th} | | | | | | |
| dose (μg/mL) | | | | | | |
| Simulated median | 472 | (144-809)^{d} | 563 | (177-1063)^{d} | 662 | (315-1037)^{e} |
| (5^{th}-95^{th} percentiles) | | | | | | |
| max C_{trough} after 8^{th} | | | | | | |
| dose (μg/mL) | | | | | | |
| Simulated median | 688 | (369-1061)^{d} | 592 | (234-1114)^{d} | 729 | (390-1105)^{e} |
| (5^{th}-95^{th} percentiles) | | | | | | |
| Cₘₐₓ after 8^{th} dose | | | | | | |
| (μg/mL) | | | | | | |
| Simulated median | 4019 | (1740-6370)^{d} | 4017 | (1515-7564)^{d} | 4855 | (2562-7522)^{e} |

(5^{th}-95^{th} percentiles)
AUC_{0-7days} after 8^{th}
dose (μg/mL•day)

^{a}Geometric mean ratio between 1,800 mg SC and 16 mg/kg was 108% (90% CI: 96, 122) in patients with multiple myeloma
^{b}Source: MMY3012 Primary Analysis Clinical Study Report
^{c}Source: AMY3001 Primary Analysis Clinical Study Report
^{d}Source: Population Pharmacokinetics and Exposure-response Analysis Report for Subcutaneously Administered Daratumumab in Multiple Myeloma Subjects
^{e}Source: Population Pharmacokinetics and Exposure-response Analysis Report for Daratumumab Subcutaneous Administration for the Treatment of Subjects with AL Amyloidosis

### Absorption

At the recommended dose of DARZALEX FASPRO 1,800 mg/30,000 units, the absolute bioavailability is 69%, with peak concentrations occurring around 3 days (Tₘₐₓ) in patients with multiple myeloma. Peak concentrations occurred around 4 days in patients with light chain (AL) amyloidosis.

### Distribution

The estimated mean (coefficient of variation, CV) volume of distribution for the central compartment is 5.2 L (37%) and peripheral compartment was 3.8 L in patients with multiple myeloma. The estimated mean volume of distribution was 10.8 L (28%) in patients with light chain (AL) amyloidosis.

### Elimination

Daratumumab is cleared by parallel linear and nonlinear saturable target mediated clearances. The estimated mean (CV %) linear clearance of daratumumab is 119 mL/day (59%) in patients with multiple myeloma and is 210 mL/day (42%) in patients with light chain (AL) amyloidosis. The estimated mean (CV %) elimination half-life associated with linear clearance is 20 days (22%) in patients with multiple myeloma and 28 days (74%) in patients with light chain (AL) amyloidosis.

### Specific Populations

The following population characteristics have no clinically meaningful effect on the pharmacokinetics of daratumumab in patients administered DARZALEX FASPRO as monotherapy or as combination therapy: sex, age (33 to 92 years), renal impairment [Creatinine clearance (CLcr) 15 to 89 mL/min as determined by the Cockcroft-Gault formula], and mild hepatic impairment (total bilirubin 1 to 1.5 times ULN and AST>ULN). The effect of moderate and severe hepatic impairment on daratumumab pharmacokinetics is unknown.

### Racial or Ethnic Groups

Of 190 patients with light chain (AL) amyloidosis who received DARZALEX FASPRO and had a maximum C_{trough} after the 8^{th} dose, African-Americans (4%) had 24% higher daratumumab mean maximum C_{trough} after the 8^{th} dose compared to that of Whites (83%) and Asians (10%) had 16% higher mean maximum C_{trough} after the 8^{th} dose compared to that of Whites. The difference in exposure between that of Asians and Whites could be explained in part by differences in body size. The effect of African-American race on exposure and related safety and efficacy of daratumumab is unknown.

### Body Weight

In patients with multiple myeloma who received DARZALEX FASPRO 1,800 mg/30,000 units as monotherapy, the mean maximum C_{trough} after the 8^{th} dose was 12% lower in the higher body weight (BW) group (>85 kg), while the mean maximum C_{trough} after the 8^{th} dose was 81% higher in the lower BW group (≤50 kg) compared to the corresponding BW groups in the intravenous daratumumab arm.

In patients with light chain (AL) amyloidosis who received DARZALEX FASPRO 1,800 mg/30,000 units in combination and had a maximum C_{trough} after the 8^{th} dose, the mean maximum C_{trough} after the 8^{th} dose was 22% lower in the higher BW group (>85 kg), while the mean maximum C_{trough} was 37% higher in the lower BW group (≤50 kg) compared to the patients with body weight of 51-85 kg.

### 13 NONCLINICAL TOXICOLOGY

### 13.1 Carcinogenesis, Mutagenesis, Impairment of Fertility

No carcinogenicity or genotoxicity studies have been conducted with daratumumab. No animal studies have been performed to evaluate the potential effects of daratumumab on reproduction or development, or to determine potential effects on fertility in males or females.

No carcinogenicity, genotoxicity, or fertility studies were conducted for recombinant human hyaluronidase. There were no effects on reproductive tissues and function and no systemic exposure of hyaluronidase in monkeys given 22,000 U/kg/week subcutaneously (12 times higher than the human dose) for 39 weeks. As hyaluronidase is a recombinant form of the endogenous human hyaluronidase, no carcinogenicity, mutagenesis, or effects on fertility are expected.

### 14 CLINICAL STUDIES

### 14.1 Newly Diagnosed Multiple Myeloma

In Combination with Bortezomib, Melphalan and Prednisone

The efficacy of DARZALEX FASPRO with bortezomib, melphalan and prednisone was evaluated in a single-arm cohort of PLEIADES (NCT03412565), a multi-cohort, open-label trial. Eligible patients were required to have newly diagnosed multiple myeloma who are ineligible for transplant. Patients received DARZALEX FASPRO 1,800 mg/30,000 units administered subcutaneously once weekly from weeks 1 to 6, once every 3 weeks from weeks 7 to 54 and once every 4 weeks starting with week 55 until disease progression or unacceptable toxicity; bortezomib 1.3 mg/m² subcutaneously twice weekly on Weeks 1, 2, 4 and 5 for the first 6-week cycle (Cycle 1; 8 doses), followed by once weekly on Weeks 1, 2, 4 and 5 for eight more 6-week cycles (Cycles 2-9; 4 doses per cycle); and melphalan 9 mg/m² and prednisone 60 mg/m² orally on Days 1 to 4 of the nine 6-week cycles (Cycles 1-9). The major efficacy outcome measure was overall response rate (ORR).

A total of 67 patients received DARZALEX FASPRO with VMP. The median age was 75 years (range: 66 to 86); 46% were male; 69% were White, 8% Asian, and 2% Black or African American; and 33% had ISS Stage I, 45% had ISS Stage II, and 22% had ISS Stage III disease.

Efficacy results are summarized in Table 17.

TABLE 17
Efficacy Results from PLEIADES in Patients Who Received DARZALEX
FASPRO-VMP

| | DARZALEX |
|---|---|
| | FASPRO-VMP |
| | (N = 67) |
| Overall response rate (sCR + CR + VGPR + PR), n (%)^{a} | 59 (88%) |
| 95% CI (%) | (78%, 95%) |
| Stringent complete response (sCR) | 5 (8%) |
| Complete response (CR) | 7 (10%) |
| Very good partial response (VGPR) | 31 (46%) |
| Partial response (PR) | 16 (24%) |

CI = confidence interval
^{a}Based on treated patients

### 14.2 Relapsed/Refractory Multiple Myeloma

In Combination with Lenalidomide and Dexamethasone

The efficacy of DARZALEX FASPRO with lenalidomide and dexamethasone (DARZALEX FASPRO-Rd) was evaluated in a single-arm cohort of PLEIADES (NCT03412565), a multi-cohort, open-label trial. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity with lenalidomide 25 mg once daily orally on Days 1-21 of each 28-day cycle; and dexamethasone 40 mg per week (or a reduced dose of 20 mg per week for patients >75 years or BMI <18.5). The major efficacy outcome measure was ORR.

A total of 65 patients received DARZALEX FASPRO with Rd. The median age was 69 years (range: 33 to 82 years); 69% were male; 69% were White, and 3% Black or African American; and 42% had ISS Stage I, 30% had ISS Stage II, and 28% had ISS Stage III disease. Patients had received a median of 1 prior line of therapy. A total of 52% of patients had a prior ASCT; 95% of patients received a prior PI; 59% received a prior immunomodulatory agent, including 22% who received prior lenalidomide; and 54% of patients received both a prior PI and immunomodulatory agent.

Efficacy results are summarized in Table 18.

TABLE 18
Efficacy Results from PLEIADES in Patients Who Received DARZALEX
FASPRO-Rd

| | DARZALEX |
|---|---|
| | FASPRO-Rd |
| | (N = 65) |
| Overall response rate (sCR + CR + VGPR + PR), n (%)^{a} | 59 (91%) |
| 95% CI (%) | (81%, 97%) |
| Stringent complete response (sCR) | 4 (6%) |
| Complete response (CR) | 8 (12%) |
| Very good partial response (VGPR) | 30 (46%) |
| Partial response (PR) | 17 (26%) |

CI = confidence interval
^{a}Based on treated patients

In Combination with Pomalidomide and Dexamethasone

The efficacy of DARZALEX FASPRO with pomalidomide and dexamethasone (DARZALEX FASPRO-Pd) versus pomalidomide and dexamethasone (Pd) alone was evaluated in APOLLO (NCT03180736), an open-label, randomized, active-controlled trial. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity with pomalidomide 4 mg once daily orally on Days 1-21 of each 28-day cycle; and dexamethasone 40 mg per week (or a reduced dose of 20 mg per week for patients >75 years). The major efficacy outcome measure was progression-free survival (PFS).

A total of 304 patients were randomized: 151 to the DARZALEX FASPRO-Pd arm and 153 to the Pd arm. The median age was 67 years (range: 35 to 90); 53% were male and 89% were White, <1% were Black or African American, and <1% were Asian, and 45% had ISS Stage I, 33% had ISS Stage II, and 22% had ISS Stage III disease. Patients had received a median of 2 prior lines of therapy (range 1-5), with 11% of patients having received 1 prior line of therapy and 75% of patients having received 2-3 prior lines of therapy. All patients received a prior treatment with a PI and lenalidomide, and 56% of patients received prior ASCT. The majority of patients were refractory to lenalidomide (80%), a PI (48%), or both an immunomodulatory agent and a PI (42%).

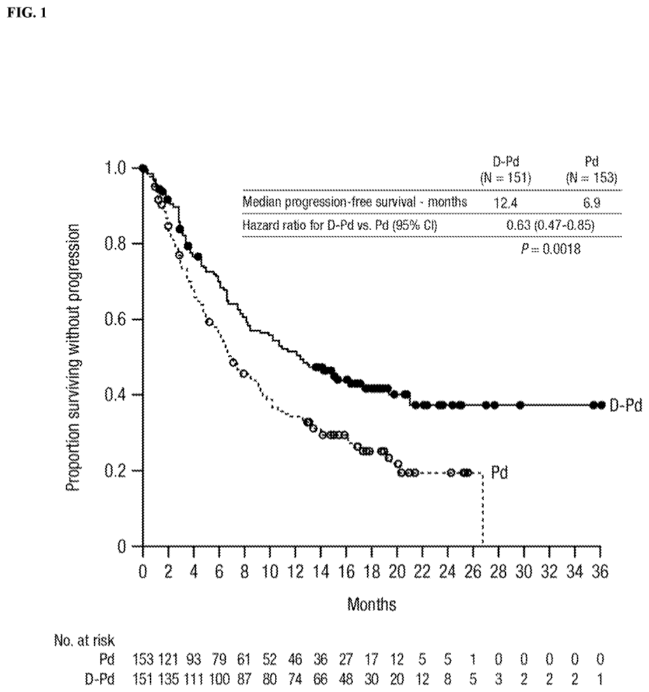

APOLLO demonstrated an improvement in PFS in the DARZALEX FASPRO-Pd treatment group as compared to the Pd treatment group; the median PFS was 12.4 months in the DARZALEX FASPRO-Pd treatment group and 6.9 months in the Pd treatment group (HR [950 CI]: 0.63 [0.47, 0.85]; p-value=0.0018), representing a 37% reduction in the risk of disease progression or death for patients treated with DARZALEX FASPRO-Pd versus Pd. See FIG. 1.

Additional efficacy results from APOLLO are presented in Table 19.

TABLE 19
Efficacy results from APOLLO^{a}

| | DARZALEX | | | |
|---|---|---|---|---|
| | FASPRO-Pd | | | |
| | (n = 151) | | Pd (n = 153) | |
| Overall response (sCR + CR + VGPR + PR) n (%)^{a} | 104 | (68.9%) | 71 | (46.4%) |
| P-value^{b} | <0.0001 | | | |
| Stringent complete response (sCR) | 14 | (9.3%) | 2 | (1.3%) |
| Complete response (CR) | 23 | (15.2%) | 4 | (2.6%) |
| Very good partial response (VGPR) | 40 | (26.5%) | 24 | (15.7%) |
| Partial response (PR) | 27 | (17.9%) | 41 | (26.8%) |
| MRD negativity rate^{c,e} n (%) | 13 | (8.6%) | 3 | (2.0%) |
| 95% CI (%) | (4.7%, 14.3%) | | (0.4%, 5.6%) | |
| P-value^{d} | 0.0102 | | | |
| MRD negativity rate in patients with CR or better^{e} | | | | |
| Number of patients with CR or better | N = 37 | | N = 6 | |
| MRD negativity rate n (%) | 13 | (35.1%) | 3 | (50.0%) |

| | | |
|---|---|---|
| 95% CI (%) | (20.2%, 52.5%) | (11.8%, 88.2%) |

Pd = pomalidomide-dexamethasone; MRD = minimal residual disease; CI = confidence interval
^{a}Based on intent-to-treat population
^{b}p-value from Cochran Mantel-Haenszel Chi-Squared test adjusted for stratification factors
^{c}Based on the intent-to-treat population
^{d}p-value from Fisher’s exact test.
^{e}Based on threshold of 10⁻⁵ using a next-generation sequencing assay (clonoSEQ).

In responders, the median time to response was 1 month (range: 0.9 to 9.1 months) in the DARZALEX FASPRO-Pd group and 1.9 months (range: 0.9 to 17.3 months) in the Pd group. The median duration of response had not been reached in the DARZALEX FASPRO-Pd group (range: 1 to 34.9+ months) and was 15.9 months (range: 1+ to 24.8 months) in the Pd group.

With a median follow-up of 16.9 months, 99 deaths were observed; 48 in the DARZALEX FASPRO-Pd group and 51 in the Pd group. Median OS was not reached for either treatment group.

### Monotherapy

The efficacy of DARZALEX FASPRO as monotherapy was evaluated in COLUMBA (NCT03277105), an open-label, randomized, non-inferiority study. Eligible patients were required to have relapsed or refractory multiple myeloma who had received at least 3 prior lines of therapy including a proteasome inhibitor and an immunomodulatory agent or who were double-refractory to a proteasome inhibitor and an immunomodulatory agent. Patients were randomized to receive DARZALEX FASPRO 1,800 mg/30,000 units administered subcutaneously or daratumumab 16 mg/kg administered intravenously; each administered once weekly from weeks 1 to 8, once every 2 weeks from weeks 9 to 24 and once every 4 weeks starting with week 25 until unacceptable toxicity or disease progression. The major efficacy outcome measures were ORR by the IMWG response criteria and maximum C_{trough} at pre-dose Cycle 3 Day 1 [see Clinical Pharmacology (12.3)]. Randomization was stratified by body weight, myeloma type, and number of prior lines of therapy.

A total of 522 patients were randomized: 263 to the DARZALEX FASPRO arm and 259 to the intravenous daratumumab arm. The median age was 67 years (range: 33 to 92 years); 55% were male; and 78% were White, 14% Asian, and 3% Black or African American. The median weight was 73 kg (range: 29 to 138). Patients had received a median of 4 prior lines of therapy. A total of 51% of patients had a prior ASCT; 100% of patients received both a PI and an immunomodulatory agent. Forty-nine percent of patients were refractory both a PI and an immunomodulatory agent. Eighty-two percent of patients were refractory to their last line of prior systemic therapy.

The results show that DARZALEX FASPRO 1,800 mg/30,000 units administered subcutaneously is non-inferior to daratumumab 16 mg/kg administered intravenously in terms of ORR and maximum trough concentration [see Clinical Pharmacology (12.3)]. Median progression-free survival was 5.6 months in the DARZALEX FASPRO arm and 6.1 months in the intravenous daratumumab arm. ORR results are provided in Table 20.

TABLE 20
Efficacy Results from COLUMBA

| | | | Intravenous | |
|---|---|---|---|---|
| | DARZALEX FASPRO | | Daratumumab | |
| | (N = 263) | | (N = 259) | |
| Overall response (sCR + CR + VGPR + PR), | 108 | (41%) | 96 | (37%) |
| n (%)^{a} | | | | |
| 95% CI (%) | (35%, 47%) | | (31%, 43%) | |
| Ratio of response rates (95% CI) | | | 1.11 | (0.89, 1.37) |
| CR or better, n (%) | 5 | (1.9%) | 7 | (2.7%) |
| Very good partial response (VGPR) | 45 | (17%) | 37 | (14%) |
| Partial response (PR) | 58 | (22%) | 52 | (20%) |

^{a}Based on intent-to-treat population.

### 14.3 Light Chain Amyloidosis

In Combination with Bortezomib, Cyclophosphamide and Dexamethasone

The efficacy of DARZALEX FASPRO with VCd was evaluated in ANDROMEDA (NCT03201965), an open-label, randomized, active-controlled trial. Eligible patients were required to have newly diagnosed light chain (AL) amyloidosis with at least one affected organ, measurable hematologic disease, Cardiac Stage I-IIIA (based on European Modification of Mayo 2004 Cardiac Stage), and NYHA Class I-IIIA. Patients with NYHA Class IIIB and IV were excluded. Patients were randomized to receive bortezomib 1.3 mg/m² administered subcutaneously, cyclophosphamide 300 mg/m² (max dose 500 mg) administered orally or intravenously, and dexamethasone 40 mg (or a reduced dose of 20 mg for patients >70 years or body mass index <18.5 or who have hypervolemia, poorly controlled diabetes mellitus or prior intolerance to steroid therapy) administered orally or intravenously on Days 1, 8, 15, and 22 of each 28-day cycle with or without DARZALEX FASPRO 1,800 mg/30,000 units subcutaneously once weekly from weeks 1 to 8, once every 2 weeks from weeks 9 to 24 and once every 4 weeks starting with week 25 until disease progression or a maximum of two years. When DARZALEX FASPRO and dexamethasone were administered on the same day, dexamethasone 20 mg was administered before DARZALEX FASPRO with the remaining dose of dexamethasone administered after DARZALEX FASPRO if applicable. The major efficacy outcome measure was confirmed hematologic complete response (HemCR) rate based on Consensus Criteria as determined by the Independent Review Committee (negative serum and urine immunofixation, involved free light chain level decrease to less than the upper limit of normal, and normal free light chain ratio). Randomization was stratified by Cardiac Stage (European Modification of Mayo 2004 Cardiac Stage) countries that typically offer autologous stem cell transplant (ASCT) for patients with light chain (AL) amyloidosis, and renal function.

A total of 388 patients were randomized: 195 to DARZALEX FASPRO-VCd and 193 to VCd. The median patient age was 64 years (range: 34 to 87 years); 58% were male; 76% White, 17% Asian, and 3% Black or African American; 23% had light chain (AL) amyloidosis Cardiac Stage I, 40% had Stage II, and 37% had Stage IIIA. The median number of organs involved was 2 (range: 1-6) and 66% of patients had 2 or more organs involved. Vital organ involvement was: cardiac 71%, renal 59% and hepatic 8%. The majority (79%) of patients had lambda free light chain disease.

Efficacy results are summarized in Table 21.

TABLE 21
Efficacy results from ANDROMEDA^{a}

| | DARZALEX FASPRO- | |
|---|---|---|
| | VCd | VCd |
| | (n = 195) | (n = 193) |
| Hematologic complete response (HemCR), n (%) | 82 (42%) | 26 (13%) |
| p-value^{b} | <0.0001 | |
| Very good partial response (VGPR), n (%) | 71 (36%) | 69 (36%) |
| Partial response (PR), n (%) | 26 (13%) | 53 (27%) |
| Hematologic VGPR or better (HemCR + VGPR), n | 153 (78%) | 95 (49%) |
| (%) | | |

| | |
|---|---|
| Major organ deterioration progression-free survival^{c}, | 0.58 (0.37, 0.92) |
| Hazard ratio with 95% CI | |

VCd = bortezomib-cyclophosphamide-dexamethasone
^{a}Based on intent-to-treat population
^{b}p-value from Cochran Mantel-Haenszel Chi-Squared test.
^{c}Major organ deterioration-PFS defined as hematologic progression, major organ (cardiac or renal) deterioration or death

The median time to HemCR was 59 days (range: 8 to 299 days) in the DARZALEX FASPRO-VCd arm and 59 days (range: 16 to 340 days) in the VCd arm. The median time to VGPR or better was 17 days (range: 5 to 336 days) in the DARZALEX FASPRO-VCd arm and 25 days (range: 8 to 171 days) in the VCd arm. The median duration of HemCR had not been reached in either arm.

The median follow-up for the study is 11.4 months. Overall survival (OS) data were not mature. A total of 56 deaths were observed [N=27 (13.8%) DARZALEX FASPRO-VCd vs. N=29 (15%) VCd group].

### 15 REFERENCES

1. Chapuy, C I, R T Nicholson, M D Aguad, et al., 2015, Resolving the daratumumab interference with blood compatibility testing, Transfusion, 55:1545-1554 (accessible at http://onlinelibrary.wiley.com/doi/10.1111/trf.13069/epdf).

### 16 HOW SUPPLIED/STORAGE AND HANDLING

DARZALEX FASPRO® (daratumumab and hyaluronidase-fihj) injection is a sterile, preservative-free, colorless to yellow, and clear to opalescent solution for subcutaneous use supplied as individually packaged single-dose vials providing 1,800 mg of daratumumab and 30,000 units of hyaluronidase per 15 mL (NDC 57894-503-01).

Store DARZALEX FASPRO vials in a refrigerator at 2° C. to 8° C. (36° F. to 46° F.) in the original carton to protect from light.

Do not freeze or shake.

### 17 PATIENT COUNSELING INFORMATION

Advise the patient to read the FDA-approved patient labeling (Patient Information).

### Hypersensitivity and Other Administration Reactions

Advise patients to seek immediate medical attention for any of the following signs and symptoms of systemic administration-related reactions: itchy, runny or blocked nose; chills, nausea, throat irritation, cough, headache, shortness of breath or difficulty breathing [see Warnings and Precautions (5.1)].

Cardiac Toxicity in Patients with Light Chain (AL) Amyloidosis

Advise patients to immediately contact their healthcare provider if they have signs or symptoms of cardiac adverse reactions [see Warnings and Precautions (5.2)].

### Neutropenia

Advise patients to contact their healthcare provider if they have a fever [see Warnings and Precautions (5.3)].

### Thrombocytopenia

Advise patients to contact their healthcare provider if they have bruising or bleeding [see Warnings and Precautions (5.4)].

### Embryo-Fetal Toxicity

Advise pregnant women of the potential hazard to a fetus. Advise females of reproductive potential to inform their healthcare provider of a known or suspected pregnancy [see Warnings and Precautions (5.5), Use in Specific Populations (8.1, 8.3)].

Advise females of reproductive potential to avoid becoming pregnant during treatment with DARZALEX FASPRO and for 3 months after the last dose [see Use in Specific Populations (8.1, 8.3)].

Advise patients that lenalidomide, thalidomide and pomalidomide have the potential to cause fetal harm and have specific requirements regarding contraception, pregnancy testing, blood and sperm donation, and transmission in sperm. Lenalidomide, thalidomide and pomalidomide are only available through a REMS program [see Use in Specific Populations (8.1, 8.3)].

Interference with Laboratory Tests

Advise patients to inform their healthcare provider, including personnel at blood transfusion centers, that they are taking DARZALEX FASPRO, in the event of a planned transfusion [see Warnings and Precautions (5.6)].

Advise patients that DARZALEX FASPRO can affect the results of some tests used to determine complete response in some patients and additional tests may be needed to evaluate response [see Warnings and Precautions (5.7)].

### Hepatitis B Virus (HBV) Reactivation

Advise patients to inform healthcare providers if they have ever had or might have a hepatitis B infection and that DARZALEX FASPRO could cause hepatitis B virus to become active again [see Adverse Reactions (6.1)].

### Product of Switzerland

We claim:

1. A method of improving progression-free survival (PFS) in an adult patient with multiple myeloma (MM), the method comprising administering to the adult patient an approved drug product comprising daratumumab and hyaluronidase in combination with pomalidomide and dexamethasone.

2. The method of claim 1, wherein the approved drug product is administered subcutaneously.

3. The method of claim 2, wherein the MM is relapsed or refractory to at least one prior line of therapy.

4. The method of claim 3, wherein the at least one prior line of therapy includes lenalidomide and a proteasome inhibitor.

5. The method of claim 4, wherein the proteasome inhibitor is bortezomib.

6. The method of claim 1, wherein the the daratumumab and hyaluronidase are administered weekly for 8 weeks, followed by administration every two weeks for eight weeks, followed by administration every 4 weeks until disease progression or unacceptable toxicity.

7. The method of claim 1, wherein the daratumumab is administered at a dose of about 1800 mg and the hyaluronidase is administered at a dose of about 30,000 Units.

8. The method of claim 1, wherein the pomalidomide is administered at a dose of 4 mg once daily on days 1-21 of each 28-day cycle, and the dexamethasone is administered at a dose of 20 to 40 mg weekly.

9. The method of claim 1 further comprising selling an approved drug product comprising daratumumab and hyaluronidase, wherein a drug product label for a reference listed drug for such drug product includes instructions for treating MM.

10. The method of claim 9, wherein the drug product is a biosimilar drug product, a Biologic License Application drug product or a supplemental Biologic License Application drug product.

11. A method of offering for sale an approved drug product comprising daratumumab and hyaluronidase, the method comprising offering for sale such drug product, wherein a drug product label for a reference listed drug for such drug product includes instructions for treating MM.

12. The method of claim 11, wherein the drug product is a biosimilar drug product, a Biologic License Application drug product or a supplemental Biologic License Application drug product.

13. A method of selling an approved drug product comprising daratumumab and hyaluronidase, the method comprising selling such drug product, wherein the drug product label for a reference listed drug for such drug product comprises PFS data.

14. A method of offering for sale an approved drug product comprising daratumumab and hyaluronidase, said method comprising offering for sale such drug product, wherein the drug product label for a reference listed drug for such drug product comprises PFS data.

15. A method of treating multiple myeloma (MM) comprising administering an approved drug product comprising daratumumab and hyaluronidase subcutaneously to an adult patient with MM in an amount that is described in a drug product label for the drug product.

16. The method of claim 13, 14 or 15, wherein the approved drug product is a biosimilar drug product, a Biologic License Application drug product or a supplemental Biologic License Application drug product.

17. The method of claim 16, wherein the wherein the drug product is administered in combination with pomalidomide and dexamethasone.

18. The method of claim 16, wherein the MM is relapsed or refractory to at least one prior line of therapy.

19. The method of claim 16, wherein the at least one prior line of therapy includes lenalidomide and a proteasome inhibitor.

20. The method of claim 16, wherein the at least one prior line of therapy includes lenalidomide and a proteasome inhibitor.

21. The method of claim 16, wherein the proteasome inhibitor is bortezomib.

22. The method of claim 16, wherein a drug product label for a reference listed drug for such approved drug product comprises PFS data.

23. The method of claim 16, wherein a drug product label for a reference listed drug for such approved drug product includes instructions for treating MM.

24. The method of claim 16, further comprising selling such approved drug product, wherein a drug product label for a reference listed drug for such approved drug product includes instructions for treating MM.

25. The method of claim 24, wherein the drug product label comprises PFS data.
